SEQUENCE LISTING (1 sequence)

SEQ ID NO: 1 - protein, 17 aa, Gallus gallus
ISQAVHAAHAEINEAGR

(19) United States
(12) Patent Application Publication
(10) Pub. No.: US 2008/0317724 A1
(43) Pub. Date: Dec. 25, 2008

(54) Micropatterned T cell stimulation

(76) Inventors: Lance Kam, New York, NY (US); Keyue Shen, New York, NY (US); Michael L. Dustin, New York, NY (US); Vedra Kaye Thomas, New York, NY (US)

Correspondence Address: SCHWEGMAN, LUNDBERG & WOESSNER, P.A., P.O. BOX 2938, MINNEAPOLIS, MN, 55402, US

(21) Appl. No.: 12/009,633

(22) Filed: Jan. 18, 2008

Related U.S. Application Data
(60) Provisional application No. 60/881,085, filed on Jan. 18, 2007.

(51) Int. Cl. A61K 35/12; C12M 1/00; A61P 31/00; C12N 5/06

(52) U.S. Cl. 424/93.71; 435/283.1; 435/372.3

(57) ABSTRACT

The invention relates to methods of expanding, stimulating or activating T cells by modulating the spatial organization of signal molecules presented to the T cells.

This application claims benefit of the filing date of U.S. Provisional Ser. No. 60/881,085, filed Jan. 18, 2007, the contents of which are specifically incorporated herein by reference.

### GOVERNMENT SUPPORT

The invention described herein was made with United States Government support under Grant Number PN2 EY016586 awarded by the National Institutes of Health through the NIH Roadmap for Medical Research. The United States Government has certain rights in this invention.

### FIELD OF THE INVENTION

The present invention relates to the control and generation of different T cell types from naïve T cells.

### BACKGROUND OF THE INVENTION

T cells are a powerful regulatory component of the immune system with potential for treatment of a wide range of diseases. T cells originate from hematopoietic stem cells in the bone marrow, then populate the thymus and expand by cell division to generate a large population of immature thymocytes. The earliest thymocytes express neither CD4 nor CD8, and are therefore classed as double-negative (CD4⁻CD8⁻) cells. As they progress through their development they become double-positive thymocytes (CD4⁺CD8⁺), and finally mature to single-positive (CD4⁺CD8⁻ or CD4⁻CD8⁺) thymocytes that are then released from the thymus to peripheral tissues. About 98% of thymocytes die during the development processes in the thymus by failing either positive selection or negative selection. Therefore, only about 2% of thymocytes survive and leave the thymus to become mature immunocompetent T cells.

Following maturation, these naïve T cells, in response to their extracellular environment and communication with other cells, differentiate into a range of classes, including effector, memory, regulatory, Th1/Th2, or senescent T cells. Each class has very specific roles in the immune system. For example, effector T cells coordinate and/or carry out the immune response to a challenge, while memory cells provide the ability to mount a fast response to later encounter with the same antigen. The relative balance of the various classes and subclasses of these cells has widespread effects on organism function.

Given the power of the immune system to identify and respond to a wide range of biomolecules, control over immune responses is emerging as a powerful tool for addressing a wide range of biomedical challenges, including treatment of distributed diseases, such as metastatic cancers. In addition, infections and diseases (e.g., human immunodeficiency viral (HIV) infections and/or cancer) can deplete T cell populations to such an extent that patients' immune systems become compromised. The patients are then vulnerable to infection. Moreover, simply having a functional immune system does not provide absolute protection from all foreign substances. For example, some viral infections (e.g., HIV), although foreign, normally do not elicit an immune response, nor do tumor cells, which are simply the organism's own cells that have lost control over their proliferation.

While there is significant interest in manipulating T cells to treat a range of diseases, current approaches cannot optimally do so. Current approaches apply stimulatory molecules on beads, and in solution, to T cells, in an effort to expand a collection of cells, such as that from circulating blood, possibly those cells that respond to a specific antigen. However, such approaches do not specifically generate significant numbers of desirable T cell types, for example, significant numbers of memory and/or effector cells are generally lacking from currently available T cell preparations. Moreover, currently available methods cannot optimally control the stimulation process.

Thus, improved methods for expanding T cell populations and controlling the phenotype of T cells are desired.

### SUMMARY OF THE INVENTION

The invention relates to methods and devices for amplifying and modulating the development and activation of specific types of T cells from naïve T cells. According to the invention, mere presentation of signal molecules to T cells does not optimally control or stimulate T cell responses. Instead, the spatial organization of the signal molecules dramatically affects T cell expansion, selection and/or activation. Thus, the inventors have discovered that the spacing, distribution and pattern of multiple signal molecules can influence cytokine secretion by T cells, a powerful indicator of cell activation and further differentiation. The methods and devices of the invention are therefore designed to utilize microscale patterning of biomolecules to direct subsequent differentiation and functioning of T cells.

Therefore, one aspect of the invention is a device for modulating T cells comprising a substrate and spatially organized predefined regions, each predefined region comprising signal molecules for modulating T cells, wherein the spatial organization of the predefined regions is optimized for modulation of T cell expansion, selection and/or activation. In some embodiments, the device has two or more different types of predefined regions where each region presents different compositions of signaling molecules, and where the predefined regions are spatially patterned and arranged to optimally modulate naïve T cells to promote development of a specific T cell function or T cell phenotype. In addition, the substrate of the device can include a coating of adhesion molecules between the predefined regions. For example, adhesion molecules such as intercellular adhesion molecule 1 (ICAM1) and/or lymphocyte function-associated antigen-1 (LFA-1) can be used between the predefined regions of the devices.

Examples of signal molecules that can used include those selected from the group consisting of type I and type II major histocompatibility complexes (both with and without an associated antigenic peptide), CCL21 (SLC/6Ckine), CD80, CD86, cytokines, CTLA-4, ICOS, Shc-Grb2-SOS, cytokines, self polypeptides, self peptides, molecules that bind and activate T cell surface antigens, or a combination thereof. T cell surface molecules that will be targeted by these molecules include T cell receptor subunits, CD3, CD28, Lck, LFA-1, CTLA-4, ICOS, or specific lipid components that influence signaling, such as those associated with lipid rafts. Cytokines that can be employed include interleukin-2, interleukin-12 and a combination thereof. In some embodiments, two or more (“costimulatory”) signal molecules are employed, for example, two or more selected from the group of signal molecules that bind to and activate CD28 (CD80/86), ICOS, CTLA-1 (CD80). Such costimulatory signal molecules can be used in conjunction with T cell receptor ligands such as anti-CD3 antibodies and/or type I and type II major histocompatibility complexes bound to antigenic peptides.

In some embodiments, the signal molecule is a self peptide or self antigen from the same individual as the T cells. In other embodiments, the signal molecule is an antigen or antigenic peptide that can stimulate the T cells. Such peptides can be used in conjunction with type I and type II major histocompatibility complexes.

In some embodiments, predefined regions have one type of signal molecule. For example, predefined regions with a first type of signal molecule can be interspersed amongst predefined regions with a second and/or third type of signal molecule. In other embodiments, one or more predefined regions has a mixture of different types of signal molecules.

The substrate employed in the devices can, for example, be silicon, glass, plastic, metal, fibrous, membrane or a combination thereof. Different types of substrates can be used in the devices, for example, chips, slides, coverslips, plates, petri dishes, microtiter wells, flasks, cylinders, particles, beads, channels, pores, crevices, protuberances or combinations thereof.

The spacing, size and geometry of predefined regions on the substrate can vary. Examples of sizes for the predefined regions include areas of about 1 μm² to about 200 μm². The signal molecules on the predefined regions can also be used in a variety of densities. For example, the signal molecules can be used in a density of about 100 to about 300 molecules/μm². The spacing between predefined regions can also vary. For example, in some embodiments the predefined regions are spaced about 0.1 to about 10 microns apart, or about 1 to 2 microns apart. Specific combinations of spacings, sizes and geometries of predefined regions are selected to promote desirable responses from naïve T cells. According to the invention, these dimensions are used not only to define the spacing, size and geometry of single types of signal molecules, but also the patterning (spacing, size and geometry) of multiple types of signal molecules that interact with different sites on the T cell, for example, both the T cell receptor and costimulatory (e.g., CD28 or CD40L) complexes. Such patterning provides better control of T cell differentiation and functioning than currently available T cell stimulatory procedures.

Another aspect of the invention is a method for modulating T cells comprising: incubating the T cells on a substrate comprising spatially organized predefined regions, each predefined region comprising signal molecules for modulating T cells, to thereby generate modulated T cells; wherein the spatial organization of the predefined regions is optimized for modulation of T cell expansion, selection and/or activation. The T cells to be modulated can be naïve T cells, for example, obtained from bone marrow, thymus, blood, lymph or lymph nodes. The T cells can, for example, be expanded or activated using the methods of the invention. In some embodiments, the T cells are activated to recognize a specific antigen. For example, the specific antigen can be a cancer antigen, viral antigen, bacterial antigen or fungal antigen. In other embodiments, T cells are manipulated to reduce reactivity to specific target antigens.

In some embodiments, the T cells are costimulated by two or more different types of signal molecules in separate predefined regions, and where the two or more types of signal molecules are present in different predefined regions that are spatially patterned and arranged to optimally modulate naïve T cells. Such modulation is used, for example, to promote naïve T cells to exhibit specific T cell functions, activities and/or phenotypes. In addition, T cell adhesion to the substrate of the device can be promoted by including a coating of adhesion molecules between the predefined regions. For example, adhesion molecules such as intercellular adhesion molecule (1 (ICAM1) and/or lymphocyte function-associated antigen-1 (LFA-1) can be used between the predefined regions of the devices.

T cells generated by the methods of the invention include helper (effector) T cells, cytotoxic T cells, memory T cells, effector T cells, regulatory T cells, natural killer T cells, γδ T cells, autoaggressive T cells and combinations thereof. In some embodiments, memory T cells or helper (effector) T cells are generated by the methods of the invention.

Another aspect of the invention is a method of treating an animal comprising administering to the animal a composition of T cells generated by incubating a T cell sample on a substrate comprising spatially organized predefined regions, each predefined region comprising signal molecules for modulating T cells, to thereby generate modulated T cells; wherein the spatial organization of the predefined regions is optimized for modulation of T cell expansion, selection and/or activation. In some embodiments, the T cell sample is from the animal (e.g., to insure that the T cells will be seen as “self” T cells). The T cell sample preferably includes naïve T cells. For example, the T cells can be obtained from bone marrow, thymus, blood, lymph or lymph nodes.

The modulated T cells are helper (effector) T cells, cytotoxic T cells, memory T cells, effector T cells, regulatory T cells, natural killer T cells, γδ T cells or autoaggressive T cells. In some embodiments, the modulated T cells are memory T cells or helper (effector) T cells.

In some embodiments, the T cells are expanded or activated. For example, the T cells can be activated to recognize a specific antigen, thereby generating “target-specific” T cells. The antigen recognized by such target-specific T cells can, for example, be a cancer antigen, viral antigen, bacterial antigen or fungal antigen.

In some embodiments, the animal to be treated by the methods of the invention has cancer. In other embodiments, the animal is infected with a virus, bacteria or fungus. For example, the animal can be infected with an immunodeficiency virus such as a human immunodeficiency virus. In further embodiments the animal is has an immune deficiency. In other embodiments, T cells are manipulated to reduce reactivity to specific targets, as part of a treatment for autoimmune or tissue rejection diseases.

Another aspect of the invention is a composition comprising an enriched population of memory or effector T cells generated by method comprising incubating a sample of T cells on a substrate comprising spatially organized predefined regions, each predefined region comprising signal molecules for modulating T cells, to thereby generate the enriched population of T cells; wherein the spatial organization of the predefined regions is optimized for T cell expansion and generation of memory or effector T cells. Such an enriched population of T cells can, for example, be an enriched population of memory or effector T cells.

### DESCRIPTION OF THE FIGURES

The patent or application file contains at least one drawing executed in color. Copies of this patent or patent application publication with color drawing(s) will be provided by the Office upon request and payment of the necessary fee;

FIG. 1A-C illustrates modulation of T cell function on micropatterned, cosignalling arrays. FIG. 1A illustrates how CD4+ cells were presented with micropatterned surfaces that capture the signaling ligands and how the microscale organization is associated with T cell costimulation. FIGS. 1B&C illustrate two specific configurations of signaling complexes, namely those intended to engage the T cell receptor and a costimulatory signals, CD28. In these demonstrations, sequential steps of microcontact printing were combined on the same surface to create these complex, multicomponent surfaces, although any of a variety of patterning techniques can be employed. FIG. 1B shows a segregated pattern containing separated regions of anti-CD3 (red) and anti-CD28 (green) antibodies, both presented within a 5-micrometer diameter circle, the size of an average naïve T cells. FIG. 1C shows a colocalized pattern created by combining the anti-CD3 and anti-CD28 antibodies (yellow) in a single patterning step. ICAM-1 was coated onto the remainder of these surfaces, but was omitted in these arrays for clarity. Scale bar=10 μm.

FIG. 2 is a series of five microscopic images illustrating migration of freshly isolated CD4+ T cells on multicomponent surfaces over time. In this time series, features of anti-CD3 (larger, 2 μm dots) and anti-CD28 (smaller, 1 μm dots) antibodies are shown in red. The cells are visible above the patterned substrate. Cells were seeded onto surfaces 30 minutes prior to collection of this time series. Scale bar=100 μm.

FIG. 3A-B illustrates one functional impact of patterned signal molecule presentation—interleukin-2 (IL-2) secretion, which is central to activation of naïve T cells, is enhanced on segregated patterns. FIG. 3A shows IL-2 secretion (green, top two panels) over 6 hours as measured in individual cells. The underlying pattern of anti-CD3 and anti-CD28 antibodies is shown in red. DIC images (bottom two panels) were used to locate all cells in the field of view. Scale bar=25 μm. FIG. 3B shows a histogram of IL-2 secretion by cells on anti-CD3-only patterns (black line, leftmost peak), anti-CD3+ anti-CD28 colocalized patterns (red line, middle peak), and anti-CD3/anti-CD28 segregated patterns (green line, diffuse, rightmost peak) from one representative experiment. IL-2 secretion for each of these conditions is statistically different from the other two (both ANOVA and Kruskal-Wallis analysis, α=0.01). Note that significantly more IL-2 fluorescence is observed when anti-CD3/anti-CD28 segregated patterns are employed (green line, diffuse, rightmost peak).

FIG. 4A-B shows that IL-2 secretion is mediated in part by NF-κB translocation. FIG. 4A shows immunofluorescently-stained NF-κB within the nucleus core as measured by segmentation of an image stack taken through each cell. FIG. 4B shows box plots of average NF-κB fluorescence in the nucleus of cells localized on anti-CD3 surfaces (CD3), on anti-CD3/anti-CD28 co-localized patterns (COL) and on anti-CD3/anti-CD28 segregated patterns (SEG). The whiskers and elements of the boxes correspond to 5, 25, 50, 75, and 95 percentiles of the data, while the diamond corresponds to the dataset average. Each condition is statistically different from the other two (α=0.01). Note that significantly more NF-κB fluorescence is observed when anti-CD3/anti-CD28 segregated patterns are employed.

FIG. 5 illustrates that the pattern of anti-CD3 and anti-CD28 antibodies does not significantly affect PKCθ localization. Patterned antibodies (both anti-CD3 and anti-CD28) are shown in red, while immunochemically detected PKCθ is shown in green. Results are for cells localized on anti-CD3 surfaces (CD3), on anti-CD3/anti-CD28 co-localized patterns (COL) and on anti-CD3/anti-CD28 segregated patterns (SEG). Cell outlines at the substrate interface were generated from long-exposure images, and are indicated by dotted lines. Scale bar=10 μm.

FIG. 6A-B shows that Akt activity may contribute to pattern recognition. FIG. 6A shows the amount of IL-2 secretion in six hours by cells incubated in the presence of the Akt-inhibitor triciribene and maintained on different patterns of anti-CD3 and anti-CD28 antibodies. In particular, cells were localized on anti-CD3 surfaces (CD3), on anti-CD3/anti-CD28 co-localized patterns (COL) and on anti-CD3/anti-CD28 segregated patterns (SEG). No significant difference was detected by either ANOVA or KW methods between experiments indicated by asterisks (α=0.05). All other comparisons indicated significant differences by KW analysis, α=0.01. FIG. 6B shows images comparing PI3K (green, showing p85α,β subunits) distribution on colocalized and segregated patterns of anti-CD3 and anti-CD28 antibodies (the patterns of these antibodies are shown in red). Cells were fixed and stained 10 minutes after seeding onto the surfaces. Scale bar=5 μm.

FIG. 7 A-C shows that IL-2 secretion correlates primarily with the CD28 geometry. FIG. 7A shows the reverse patterns of anti-CD3 and anti-CD28 ligands employed. FIG. 7B shows the relative amount of IL-2 secretion by cells over a 6 hour incubation on the reverse patterns shown in FIG. 7A compared to the segregated and colocalized pattern used in FIGS. 5 and 6. Data from each pattern is different from all other conditions by KW analysis, α=0.05. FIG. 7C shows en face projections of dendritic cells with either naïve (top row) or previously activated (bottom row) CD4+ T cells, illustrating ultifocal, segregated patterns of TCR (red) and CD80 (green) within the generated immune synapse (IS) structures. Dotted lines delineate the cell-cell interface. Scale bars=2 μm.

FIG. 8A-E illustrate different patterns of the invention and some methods for generating substrates with patterned signal molecules. FIG. 8A illustrates how materials can be layered to display selected signal molecules. For example, materials exhibiting a binding specificity for anti-CD3 (red) and/or anti-CD28 (green) are layered and pores or channels are etched through the resultant structure allow access to the pattern by T cells in a bioreactor environment. Different layers can have different affinities for different signal molecules so that a three dimensional pore, channel or crevice is available to T cells and one or more sides of the T cells can interact with the different layers. FIG. 8B illustrates entropic capture of functionalized beads on a pre-patterned surface. FIG. 8C shows some patterns of signal molecules (e.g., anti-CD3 (Y symbols) and anti-CD28 (X symbols) antibodies) that can be used on beads. The leftmost bead shows currently available beads having a mixture of anti-CD3 and anti-CD28 antibodies on beads having a diameter of about 10 microns. The beads in the middle and to the right show some embodiments of the invention that have been specifically patterned to optimally modulate T cells. The middle bead has clusters of anti-CD28 (X symbols) antibodies in a small predefined region of the bead and larger clusters of anti-CD3 (Y symbols) in a larger pre-defined region of the bead. The right bead has two predefined regions, where anti-CD3 antibodies (Y symbols) occupy one region and anti-CD28 antibodies (X symbols) occupy the other, each predefined region constituting about one-half of the area of the bead. FIG. 8D illustrates different methods for patterning beads. The left schematic shows how to make alternating features of signal molecules on beads. To generate such an alternating pattern on beads, the signal molecules can be first patterned onto a planar stamp (which is much easier than directly patterning the beads) and then the pattern of signal molecules can be applied onto the beads by rolling the beads on the stamp. The right schematic shows a bead with two predefined regions, each consisting of approximately one-half of the bead. Such a bead can be made by generating dual-functionalized surfaces on the bead. For example, one-half of a silicon or glass bead can be coated with gold by a metal sputtering process to generate beads with sold on one side (shaded area) and silica on the other side (non-shaded area). Well-established, two-component chemistries, such as selective binding of thiols to gold and silanes to silicon oxide (native layer on a silicon wafer or glass bead) can be used to generate different binding or functional groups on the two surfaces useful for capturing/immobilizing the signal molecules of interest. The patterned beads are useful for modulating T cells because they present the signal molecules to the T cells in the spatial manner used in vivo for cell-cell signaling processes. FIG. 8E illustrates methods for generating patterns on substrate surfaces, for example, on silicon surfaces. A silicon substrate (top image) is treated with photoresist to generate regions having photoresist (non-shaded raised areas) and other, open areas without photoresist. The substrate is then coated with gold (shaded areas on the third image), which will attach to the regions not treated with photoresist. Photoresist can then be applied to the edges of the gold regions to generate levies or dams (non-shaded areas flanking the gold shaded areas in the fourth image from the top) to facilitate attachment of different functional groups, binding agents and/or linkers (R₁ and R₂ groups) to the gold (with R₁ groups) and silicon (with R₂ groups) surfaces (see fifth image from the top). The selected signal molecules (X and Y symbols) can then be attached to separate predefined regions (bearing R₁ and R₂ groups, respectively).

FIG. 9A-C illustrates multicomponent patterns that can be formed on substrates to generate the devices of the invention. FIG. 9A-B show that microcontact printing can be used to create colocalized (FIG. 9A) or segregated (FIG. 9B) patterns of anti-CD3 (red) and anti-CD28 (green) antibodies. FIG. 9C illustrates patterning of three types of supported lipid bilayers using membrane fluidics.

### DETAILED DESCRIPTION OF THE INVENTION

According to the invention the spatial presentation of signal molecules can dramatically affect the response of T cells to those signal molecules. For example, when anti-CD3 and anti-CD28 antibodies are placed on separate predefined regions of a substrate, T cells incubated on the substrate secrete different amounts of interleukin-2 and/or exhibit spikes in calcium, depending not only on the types but also on the spacing of these signal molecules.

Therefore, aspects of the invention include methods and devices for modulating and/or stimulating T cell expansion, selection and/or activation. The methods and devices involve spatial presentation of signal molecules to T cells or T cell progenitor cells to modulate the expansion, selection and/or activation of those cells.

### DEFINITIONS

As used herein an “array” is a collection of signal molecules, at least two of which are different, arranged on a substrate in a spatially defined and physically addressable manner.

As used herein a “predefined region” is a predefined region in a localized area on a substrate (e.g., on an array) and is otherwise referred to herein in the alternative as a “selected” region or simply a “region.” The predefined region may have any convenient shape, e.g., circular, rectangular, linear, elliptical, wedge-shaped, etc. In some embodiments, a predefined region and, therefore, the area upon which each distinct signal molecule is placed or bound is smaller than about 200 μm², and in some embodiments the predefined regions have an area of about 1 μm to about 200 μm².

As used herein, a “feature” is a predefined region with signal molecules. Each feature can have a different type and/or density of signal molecules. Alternatively, several or many features can have the same type and/or density of signal molecules.

### Signal Molecules

T cell expansion, selection and/or activation can be modulated by a variety of molecules. As used herein, the molecules that can modulate T cell expansion, selection and/or activation are “signal molecules.” These signal molecules are spatially patterned to selectively and optimally activate specific proteins on the surface of T cells. The T cell proteins that are activated then drive desirable T cell functions. Examples of signal molecules that can be used in the methods and devices of the invention include any of the molecules involved in the in vivo processes of T cell expansion, selection and/or activation. In addition, the signal molecules of the invention can include molecules that mimic the effects of molecules involved in the in vivo processes of T cell expansion, selection and/or activation. In some embodiments, the signal molecules are natural ligands to T cell surface proteins, or molecules that mimic the effects of such natural ligands.

Examples of signal molecules that can used include those selected from the group consisting of type I and type II major histocompatibility complexes (both with and without an associated antigenic peptide), CCL21 (SLC/6Ckine), CD80, CD86, cytokines, CTLA-4, ICOS, Shc-Grb2-SOS, cytokines, self polypeptides, self peptides, molecules that bind and activate T cell surface antigens, or a combination thereof. T cell surface molecules that will be targeted by these molecules include T cell receptor subunits, CD3, CD28, Lck, LFA-1, CTLA-4, ICOS, or specific lipid components that influence signaling, such as those associated with lipid rafts. Cytokines that can be employed include interleukin-2, interleukin-12 and a combination thereof.

According to the invention, different types of signal molecules are used depending upon the starting type of T cell to be modulated and the desired T cell to be produced. In general, it is desirable to start with naïve T cells (e.g., from bone marrow, blood, lymph and/or lymph nodes). Activation of naïve T cells to generate desirable types of T cells (e.g., memory and/or effector T cells) generally requires use not only of signal molecules that bind the T cell receptor but also signal molecules that bind costimulatory molecules on the T cell.

The T cell receptor is part of a complex signaling machinery that includes the TCRαβ dimer, the CD4 or CD8 accessory molecules and a signal transduction module made up of the various chains of CD3. Engagement of the T cell receptor by a peptide antigen, in association with type I and type II major histocompatibility complexes, leads to a series of intracellular biochemical events culminating in the transcription of new genes and cellular activation. One early event is the activation of one or more tyrosine kinases that first phosphorylate CD3 polypeptide chains themselves and subsequently phosphorylate other substrates. After tyrosine kinase activation, a series of events follow T cell receptor engagement, including activation of serine/threonine kinases, activation of the GTP-binding protein p21ras, and activation of transcription factors for receptors and growth factors such as the major T cell growth factor interleukin-2. The CD4 and CD8 co-receptors bind a tyrosine kinase (p56lck) via their intracytoplasmic tail, which plays a role in T cell signaling.

However T cell receptor binding is not sufficient to optimally activate T cells. Two or more distinct costimulatory molecules exist on T cells that interact with specific ligands on the surface of antigen presenting cells (APC). Such T cell costimulatory molecules include CD28, CD40 ligand (CD40L), ICOS and OX40. The ICOS and OX 40 costimulatory molecules may act on memory cells while CD28 is predominantly a naïve T cell activator.

The CD28 molecule on T cells delivers a costimulatory signal upon engaging either of its ligands. In vivo, these CD28 ligands are B7.1 (CD80) or B7.2 (CD86). Similarly, a distinct signal is transduced by the CD40L molecule on the T cell when it is ligated to its ligand, CD40. Antigen presenting cells express the B7.1/B7.2 and CD40. According to the invention, B7.1/B7.2 and CD40 can be used as signal molecules for modulating T cells, for example, as costimulatory signal molecules. B7.1 and B7.2 can also regulate T cells by binding cytotoxic T-lymphocyte antigen (CTLA)-4, which inhibits T cell proliferation.

Thus, in some embodiments, T cell receptor signal molecules are used in conjunction with one or more (“costimulatory”) signal molecules. Such costimulatory signal molecules include, for example, those signal molecules that bind to CD28 (e.g., anti-CD28 antibodies and/or CD80/86), ICOS (e.g., anti-ICOS antibodies and/or B7-H2), T cell lymphocyte antigen-1 (CTLA-1) (e.g., CD80). Such costimulatory signal molecules can be used in conjunction with T cell receptor ligands such as anti-CD3 antibodies and/or type I and type II major histocompatibility complexes with one or more antigenic peptides.

Costimulation provides an instructive signal that regulates T cell response when the antigen is recognized. These responses are primarily associated with naïve cells, and not with previously activated cells. This is one stage in the differentiation of a T cell when the resultant T cell class (memory, effector, etc.) can be modulated. Thus, the invention provides devices and methods for generating a specific T cell class (e.g., memory or effector T cells) by simultaneously presenting a pattern of different signal molecules, where some of the signal molecules bind to and/or activate the T cell receptor and other signal molecule costimulate other T cell polypeptides (e.g., CD28 and/or CD40L).

Antibodies to a variety of T cell proteins can be used in the methods and devices of the invention. For example, the antibodies can be directed against and/or bind specifically to T cell receptor alpha and beta (TCRα and TCRβ) gene products; CD28 proteins; CD3 proteins (e.g., CD3εγ and CD3εδ heterodimers and/or the CD3ζ homodimer) or any of six ITAM motifs on CD3 polypeptide. The antibodies can also be directed against or bind to CD28, Lck, ZAP-70, CD80, CD86, Trim, LAT, SLP-76, VAV1, Itk, PI(4,5)P2, LFA-1, CTLA-4, ICOS and other T cell-associated proteins.

In addition, the a coating of adhesion molecules can be used on the substrate surface(s) between the predefined regions. For example, adhesion molecules such as intercellular adhesion molecule 1 (ICAM1), which binds to the lymphocyte function-associated antigen-1 (LFA-1) on T cells, can be used between the predefined regions of the devices.

Signal molecules can readily be obtained from a variety of sources. For example, signal molecules are generally available from GenWay Biotech, Inc. (San Diego, Calif.), Cell Signaling Technology, Inc. (Danvers, Mass.), R&D Systems (Minneapolis, Minn.), Invitrogen Corp. (Carlsbad, Calif.) and others.

### T Cell Selection

According to the invention, T cell selection processes can be mimicked, modulated and/or stimulated using the methods and devices of the invention.

T cells originate from hematopoietic stem cells in the bone marrow. Hematopoietic progenitors derived from hematopoietic stem cells populate the thymus and expand by cell division to generate a large population of immature thymocytes. The earliest thymocytes express neither CD4 nor CD8, and are therefore classed as double-negative (CD4⁻CD8⁻) cells. As they progress through their development they become double-positive thymocytes (CD4⁺CD8⁺), and finally mature to single-positive (CD4⁺CD8⁻ or CD4⁻CD8⁺) thymocytes that are then released from the thymus to peripheral tissues. About 98% of thymocytes die during the development processes in the thymus by failing either positive selection or negative selection processes described below.

Positive selection occurs as follows. Double-positive thymocytes move deep into the thymic cortex where they are presented with self-antigens (i.e., antigens that are derived from molecules belonging to the host of the T cell) complexed with MHC molecules on the surface of cortical epithelial cells. Only those thymocytes that bind the MHC/antigen complex with adequate affinity will receive a vital “survival signal.” Developing thymocytes that do not have adequate affinity cannot serve useful functions in the body; the cells must be able to interact with MHC and peptide complexes in order to affect immune responses. Therefore, the other thymocytes with low affinity die by apoptosis and their remains are engulfed by macrophages. This process is called positive selection.

Whether a thymocyte becomes a CD4+ cell or a CD8+ cell is also determined during positive selection. Double-positive cells that are positively selected on MHC class II molecules will become CD4+ cells, and cells positively selected on MHC class I molecules become CD8+ cells.

Note that this process does not remove thymocytes from the population that would cause autoimmunity or would react with mammal's own cells. The removal of such cells is dealt with by negative selection, which is discussed below.

The negative selection process occurs as follows. Thymocytes that survive positive selection migrate towards the boundary of the thymic cortex and thymic medulla. While in the medulla, they are again presented with self-antigen in complex with MHC molecules on antigen-presenting cells (APCs) such as dendritic cells and macrophages. Thymocytes that interact too strongly with the antigen receive an apoptosis signal that causes their death; the vast majority of all thymocytes initially produced end up dying during thymic selection. A small minority of the surviving cells is selected to become regulatory T cells. The remaining cells will then exit the thymus as mature naïve T cells. This negative selection process is an important mechanism for generating immunological tolerance, which prevents the formation of self-reactive T cells capable of generating autoimmune disease in the host.

### T Cell Activation

The methods and devices of the invention can be used to modulate and/or stimulate T cell activation. Any of the molecules involved in T cell activation can be used as signal molecules in the methods and devices of the invention.

Although the specific mechanisms of activation vary slightly between different types of T cells, the “two-signal model” of activation generally occurs for most CD4+ T cells. Thus, activation of CD4+ T cells occurs through the engagement of both the T cell receptor and CD28 on the T cell by the Major Histocompatibility Complex (MHC) peptide and by B7 family members on the antigen presenting cell (APC), respectively. Both are required for production of an effective immune response. In the absence of CD28 co-stimulation, T cell receptor signaling alone results in anergy.

The signaling pathways downstream from both CD28 and the T cell receptor involve many proteins. The first signal is provided by binding of the T cell receptor (TCR) to a short peptide presented by the major histocompatibility complex (MHC) on another cell. This ensures that only a T cell with a TCR specific to that peptide is activated. The partner cell is usually a professional antigen presenting cell (APC), usually a dendritic cell in the case of naïve responses, although B cells and macrophages can be important APCs. The peptides presented to CD8+ T cells by MHC class I molecules are 8-9 amino acids in length. The peptides presented to CD4+ cells by MHC class II molecules are longer, as the ends of the binding cleft of the MHC class II molecule are open.

The second signal comes from co-stimulation, in which surface receptors on the APC are induced by a relatively small number of stimuli, usually products of pathogens, but sometimes breakdown products of cells, such as necrotic-bodies or heat-shock proteins. The only co-stimulatory receptor expressed constitutively by naïve T cells is CD28, so co-stimulation for these cells comes from the CD80 and CD86 proteins on the APC. Other receptors are expressed upon activation of the T cell, such as OX40 and ICOS, but these largely depend upon CD28 for their expression. The second signal allows the T cell to respond to an antigen. Without this second signal, the T cell becomes anergic, and activation of anergic T cells in the future is difficult. This mechanism prevents inappropriate responses to self, as self-peptides will not usually be presented with suitable co-stimulation.

The T cell receptor exists as a complex of several proteins. The actual T cell receptor is composed of two separate peptide chains, which are produced from the independent T cell receptor alpha and beta (TCRα and TCRβ) genes. The other proteins in the complex are the CD3 proteins: CD3εγ and CD3εδ heterodimers and, most important, a CD3ζ homodimer, which has a total of six ITAM motifs. The ITAM motifs on the CD3ζ can be phosphorylated by Lck and in turn recruit ZAP-70. Lck and/or ZAP-70 can also phosphorylate the tyrosines on many other molecules, not least CD28, Trim, LAT and SLP-76, which allows the aggregation of signaling complexes around these proteins.

Phosphorylated LAT recruits SLP-76 to the membrane, where it can then bring in PLCγ, VAV1, Itk and potentially PI3K. Both PLCγ and PI3K act on PI(4,5)P2 on the inner leaflet of the membrane to create the active intermediaries di-acyl glycerol (DAG), inositol-1,4,5-trisphosphate (IP3), and phosphatidlyinositol-3,4,5-trisphosphate (PIP3). DAG binds and activates some PKCs, most important, in T cells PKCθ, a process important for activating the transcription factors NF-κB and AP-1. IP3 is released from the membrane by PLCγ and diffuses rapidly to activate receptors on the ER, which induce the release of calcium. The released calcium then activates calcineurin, and calcineurin activates NFAT, which then translocates to the nucleus. NFAT is a transcription factor, which activates the transcription of a pleiotropic set of genes, most notable, IL-2, a cytokine that promotes long term proliferation of activated T cells.

### T Cell Types

The methods of the invention can be performed on any type of T cell. Useful sources of T cells include bone marrow, circulating blood, thymus, lymph and lymph nodes.

Several different subsets of T cells have been described, each with a distinct function. Any of these types of T cells can be made, manipulated or modulated using the methods and devices of the invention. In some embodiments, memory and/or helper (effector) T cells are preferably generated using the methods and devices of the invention.

Helper T cells (T_{H} cells, also called effector T cells) are the “middlemen” of an adaptive immune system. Once activated, they divide rapidly and secrete small proteins called cytokines that regulate or help the immune response. Depending on the cytokine signals received, these cells differentiate into T_{H}1, T_{H}2, T_{H}17, or one of other subsets, which secrete different cytokines.

Cytotoxic T cells (T_{C} cells, or CTLs) destroy virally infected cells and tumor cells, and are also implicated in transplant rejection. These cells are also known as CD8⁺ T cells, since they express the CD8 glycoprotein at their surface. Through interaction with helper T cells, these cells can be transformed into regulatory T cells, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise two subtypes: central memory T cells (T_{CM} cells) and effector memory T cells (T_{EM} cells). Memory cells may be either CD4+ or CD8+.

Regulatory T cells (T_{reg} cells), formerly known as suppressor T cells, are needed for the maintenance of immunological tolerance. Their major role is to shut down T cell-mediated immunity toward the end of an immune reaction and to suppress auto-reactive T cells that escaped the process of negative selection in the thymus. Two major classes of CD4+ regulatory T cells have been described, including the naturally-occurring T_{reg} cells and the adaptive T_{reg} cells. Naturally-occurring T_{reg} cells (also known as CD4⁺CD25⁺FoxP3⁺ T_{reg} cells) arise in the thymus, whereas the adaptive T_{reg} cells (also known as Tr1 cells or Th3 cells) may originate during a normal immune response. Naturally-occurring T_{reg} cells can be distinguished from other T cells by the presence of an intracellular molecule called FoxP3. Mutations of the FOXP3 gene can prevent regulatory T cell development, causing the fatal autoimmune disease IPEX.

Natural Killer T cells (NKT cells) are a special kind of lymphocyte that bridges the adaptive immune system with the innate immune system. Unlike conventional T cells that recognize peptide antigen presented by major histocompatibility complex (MHC) molecules, NKT cells recognize glycolipid antigen presented by a molecule called CD1d. Once activated, these cells can perform functions ascribed to both Tₕ and T_{c} cells (i.e., cytokine production and release of cytolytic/cell killing molecules).

γδ T cells represent a small subset of T cells that possess a distinct TCR on their surface. A majority of T cells have a TCR composed of two glycoprotein chains called α- and β-TCR chains. However, in γδ T cells, the TCR is made up of one γ-chain and one 6-chain. This group of T cells is much less common (5% of total T cells) than the αβ T cells, but are found at their highest abundance in the gut mucosa, within a population of lymphocytes known as intraepithelial lymphocytes (IELs). The antigenic molecules that activate γδ T cells are still widely unknown. However, γδ T cells are not MHC restricted and seem to be able to recognize whole proteins rather than requiring peptides to be presented by MHC molecules on antigen presenting cells. Some recognize MHC class IB molecules though. Human Vγ9/Vδ2 T cells, which constitute the major γδ T cell population in peripheral blood, are unique in that they specifically and rapidly respond to a small non-peptidic microbial metabolite, HMB-PP, an isopentenyl pyrophosphate precursor.

Autoaggressive T cells are a unique T cell subset that is characterized by the expression of CD40. CD40 typically is associated with antigen-presenting cells, but is also expressed on a subset of T helper cells. Th40 cells are found in all individuals but occur at drastically-expanded percentages in autoimmune subjects. This is true of autoimmune humans and mice. Th40 cells from type 1 diabetic subjects respond to known self-antigens, whereas Th40 cells from non-autoimmune subjects do not respond to those antigens. A crucial role of CD40 on T cells is to induce RAG1 and RAG2, the recombinase proteins responsible for altering the T cell receptor. The TCR is the means by which T cells are able to recognize antigens. Dogma requires that RAG1 and RAG2 be expressed only in the thymus, during T cell-development. However RAGs are re-expressed in peripheral T cells and CD40 engagement on Th40 cells induces RAGs expression. Following RAG expression changes in TCR occur. This means that Th40 cells are capable of adapting throughout an individual's lifetime. This process of altering TCR expression in the periphery is called TCR revision. Revision can be responsible for expanding the T cell repertoire, but also could result in the generation of autoaggressive T cells. TCR revision is therefore another means of T cell tolerance. Alteration of autoaggressive TCR would necessarily tolerize those T cells.

### Devices

As described and exemplified herein, the spatial arrangement of signal molecules presented on a substrate surface modulates T cell function. For example, experiments described herein demonstrate that the amount of interleukin 2 (IL-2) secreted by T cells can be varied by modulating the type and spatial organization of signal molecules presented to T cells. Thus, for example, a pattern was generated with anti-CD3 and anti-CD28 antibodies, where anti-CD3 antibodies occupied a central feature surrounded by satellite features of anti-CD28 antibodies that were spaced about 1 to 2 microns from the central anti-CD3 feature. When the anti-CD28 antibody features were spaced about 1 to 2 microns apart, the T cell secretion of interleukin-2 (IL-2) was enhanced compared to when the anti-CD3 and anti-CD28 antibodies were presented together to the T cells in “colocalized” features. Therefore, by spatially arranging signal molecules into specific patterns, T cell function and phenotype can be modulated.

Thus, one aspect of the invention is a device that includes a substrate with an array or pattern of signal molecules on the substrate. The device generally includes a solid substrate. In some embodiments, the device has two or more different types of predefined regions where each region presents different compositions of signaling molecules, and where the predefined regions are spatially patterned and arranged to optimally modulate naïve T cells. For example, the T cells can be modulated to promote development of a specific T cell function or T cell phenotype. In addition, the substrate of the device can include a coating of adhesion molecules between the predefined regions. For example, adhesion molecules such as intercellular adhesion molecule I (ICAM1) and/or lymphocyte function-associated antigen-1 (LFA-1) can be used between the predefined regions of the devices.

The signal molecules are covalently or non-covalently attached to predefined regions of the substrate. Different types of signal molecules are provided in different predefined regions of the substrate to generate an array or pattern of signal molecules. The spacing between predefined regions where the different signal molecules reside is optimized for modulation of T cell expansion, selection and/or activation.

Substrates that can be used in the invention include silicon, glass, plastic, metal, fibrous, and membrane substrates. The substrates can also assume a variety of shapes. Thus, the substrates can be substantially flat substrates such as chips, slides, coverslips, plates, Petri dishes, microtiter wells, flasks (e.g., the bottom of flasks) and other containers where cells may be grown. Alternatively, the substrates can have a three-dimensional form, for example, the substrate can include beads, particles, etched surfaces with pores, channels or crevices, rounded surfaces of a flasks or microtiter wells.

Distinct types of signal molecules are placed in predefined regions on the substrate. The signal molecules can be placed on flat portions of the substrate, or on curved portions of the substrate, as well as in pores, crevices, channels, and protuberances of the substrate.

In general, the predefined regions have an area of about 1 μm² to about 200 μm², or about 5 μm² to about 150 μm², or about 10 μm² to about 100 μm². The sizes of naïve T cells are substantially consistent across species, with a contact area measuring about 5 to 10 microns (e.g., about 7-μm) in diameter, and about 30 to 50 μm² (e.g., 40 μm²) in area. Therefore, patterning dimensions for predefined regions are often on the micrometer scale, but it is generally advantageous that many predefined regions be present over a large area so that the resultant patterns to allow production of clinically relevant numbers of cells (tens of millions). For example, the total patterned area can be fairly large (e.g., one or more square inches) while retaining a plurality of cell-scale dimensions for the predefined regions.

According to the invention, T cell activation is influenced by the surface density of signal molecules presented to the T cells, as well as the spatial organization of signal molecules. In general, signal molecule densities of about 100 to about 300 molecules/μm² are used in the predefined regions of the substrate. In some embodiments, optimal signal molecule densities of about 200 molecules/μm² plus or minus about 15% of 200 molecules/μm² are used in the predefined regions of the substrate.

The spacing between predefined regions of signal molecules also influences the response of T cells. Thus, the predefined regions are spaced about 0.1 to about 10 microns apart, or about 0.3 to about 7 microns apart, or about 0.5 to about 5 microns apart. In some embodiments, the predefined regions with signal molecules are optimally spaced about 1 to 2 microns apart.

The signal molecules displayed on the substrates in accordance with the invention may be attached directly or indirectly to a substrate using methods described herein as well as any methods available in the art.

The signal molecules can be attached or adsorbed onto the substrate using any available methods. For example, microscale patterns of proteins can be generated using methods employed by the microelectronics industry. Thus, the signal molecules can be applied or attached to the substrate using microcontact printing, soft-lithography, layering of materials containing the signal molecules, layering of materials containing molecules/moieties to which the signal molecules can be attached and entropic capturing of beads and/or particles.

However, methods that involve heating and chemical stripping steps should generally be avoided because these steps are not compatible with biological molecules—the activity and structure of many proteins can be lost under these conditions. In general, microcontact or contact printing processes are gentle enough to be used with biomolecules and are sufficiently simple, efficient, relatively inexpensive and adaptable for use with a variety of signal molecules and substrates. This method generally uses stamps to pattern the surfaces of substrates.

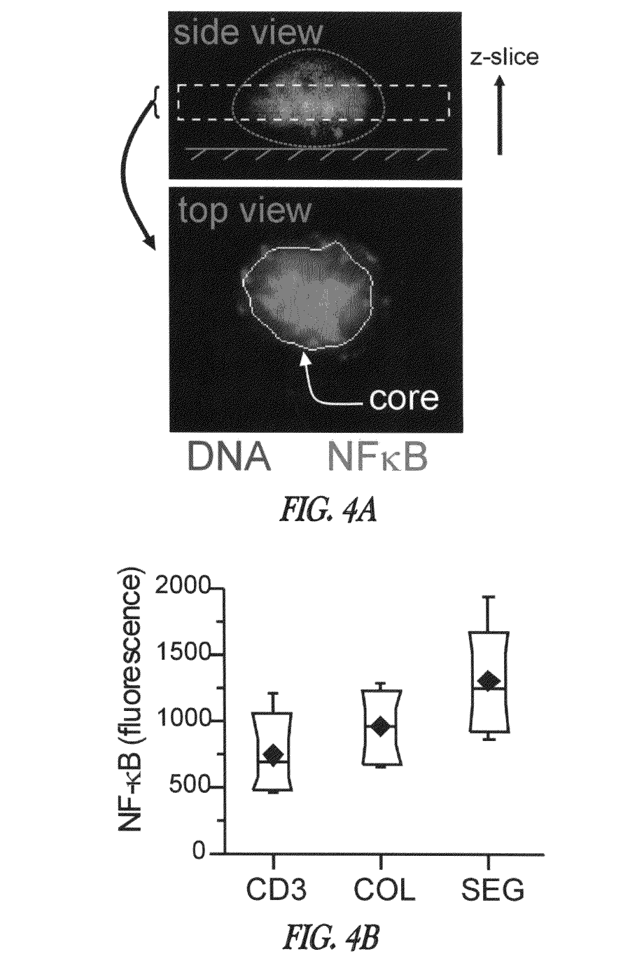

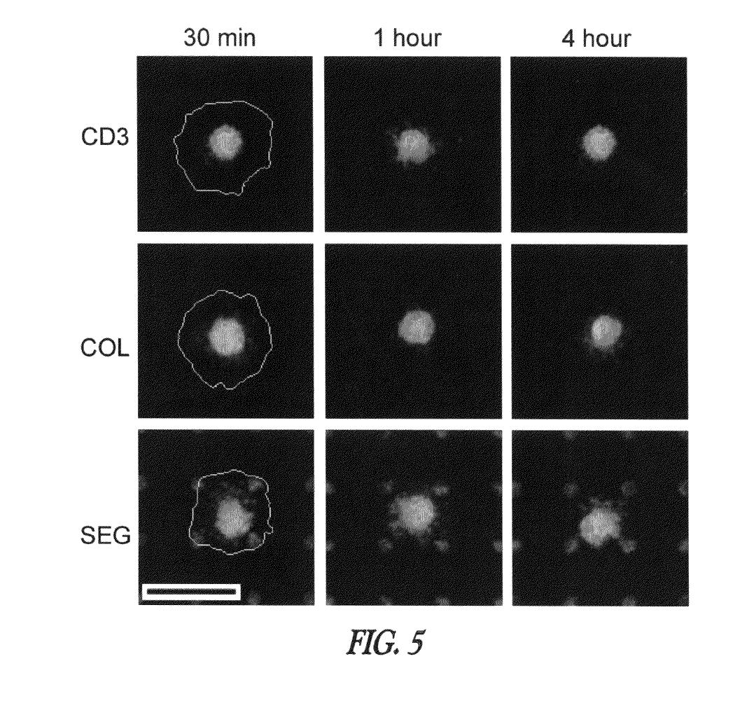

In other embodiments, nanoimprint and/or microcontact patterning and/or lithography procedures can be employed for high-throughput patterning of surfaces, including curved structures such as beads and cylinders. Such patterning methods can be used to directly pattern the signaling molecules of interest onto the surface or to deposit linking molecules that are more resistant to patterning conditions and are subsequently used to associate or link the signal molecules with the substrate. Methods for patterning biological molecules onto substrates using these types of methods are described, for example, in U.S. Pat. No. 5,776,748 and Chemiavskaya et al. (J. Vac. Sci. Technol. B 23(6): 2972-78 (2005) (see, e.g., FIG. 4 of Chemiavskaya et al.)), the contents of which publications are specifically incorporated herein in their entireties.

Briefly, a polymeric material can be cast onto a mold with raised features to define the desired pattern on a stamp. The stamp can be cured after separation from the mold, and then “inked” either with a solution of signal molecules or with a solution that facilitates binding of the signal molecules to the substrate. When the stamp is pressed against the substrate, a pattern of signal molecules is generated or a pattern of predefined regions inked with an adhesive or binding substance is generated (depending on whether the signal molecules are directly attached to the substrate or attached via a linker or other adhesive/binding substance). If a binding substance is used, the substrate is then exposed to a solution of signal molecules so that the signal molecules bind, attach, adsorb or associate with the predefined regions having the binding substance. The signal molecules do not bind to the regions not inked by the binding substance. However, the substrate can be exposed to a second or filling solution to coat the bare substrate between the predefined regions that have or will have the signal molecules. Such a second or filling solution can be used before or after the signal molecules have adhered to the substrate.

The mold used to form the stamp may be a commercially available item such as a transmission electron microscopy grid or any other corrugated material possessing a pattern which is desired to be reproduced on the stamp. Alternatively, the mold may be prepared by any of a variety of methods known in the art. For example, the mold surface can be micromachined from a material such as metal. Alternatively, the mold surface is formed lithographically by providing a mold substrate, depositing a film of material onto the mold substrate, coating an exposed surface of the material with resist, irradiating the resist according to a predetermined pattern, removing irradiated portions of the resist from the material surface, contacting the material surface with a reactant selected to react chemically therewith and selected to be chemically inert with respect to the resist such that portions of the material according to the predetermined pattern are degraded, removing the degraded portions, and removing the resist to uncover portions of the material formed according to the predetermined pattern to form the mold surface. Negative or positive resist may be used, and the procedure is adjusted accordingly. Another method of forming a mold surface involves providing a substrate and coated with resist. Then portions of the resist may be irradiated according to a particular predetermined pattern. Irradiated portions of the resist may then be removed from the substrate to expose portions of the substrate surface according to the predetermined pattern, and the substrate may be contacted with a plating reagent such that exposed portions according to the predetermined pattern are plated. Then, the resist may be removed to uncover portions of the exposed substrate according to the predetermined pattern bordered by plated portions of the substrate to form the mold surface. As noted above, any corrugated material may be used as a mold to form the stamps of the present invention.

The stamp is produced by casting a polymeric material onto a mold having the desired pattern. The particular material chosen for formation of the stamp is not critical, but is generally chosen so as to satisfy certain physical characteristics. The stamp is typically chosen to be elastic, such that the stamping surface may very closely conform to minute irregularities in the surface material of the plate to be stamped and to completely transfer the ink thereto, and so as to be amenable to transferring signal molecules to nonplanar surfaces. The stamp should not, however, be so elastic as to greatly deform in shape during stamping as this will cause a blurring of the desired pattern. The stamp should also be formed such that the stamping surface comprises an absorbent material selected to absorb signal molecule, adhesive or binding solutions. The stamp material may also be capable of swelling. Such swelling and absorbing characteristics serve to provide good definition of predefined regions on the surface material of a substrate. For example, if a predefined region of the stamping surface is substantially square-shaped, the stamping surface should transfer the signal molecule or adhesive/binding solutions to the surface material of the substrate plate to form predefined regions on the substrate that mirror the substantially square features of the stamping surface, without blurring. Such blurring results from selection of a stamp that does not absorb the “ink.” When such a stamp is employed, the ink resides as a liquid on the stamping surface, rather than partially or fully within the surface material of the stamp, and when the stamping surface contacts the surface of the substrate, the ink is dispersed from under the stamping surface.

Additionally, the stamp should be fabricated such that the stamping surface is free of any leachable materials such as plasticizers that would interfere with or contaminate the ink. For example, if additives are included in the material used to fabricate the stamp, such additives should be bound to the internal structure of the stamp. For example, if the stamp is fabricated from a polymeric material, any additives should be bound to the polymer backbone thereof.

Material selected for use in fabrication of the stamp is advantageously selected so as not to undergo substantial shape changes when the stamp is formed. One type of material that is generally suitable is a polymeric material. Polymeric materials suitable for use in the fabrication of the stamp may have linear or branched backbones, and may be crosslinked or non-crosslinked, depending upon the particular polymer and the degree of formability desired of the stamp. A variety of elastomeric polymeric materials is suitable for such fabrication, especially polymers of the general classes of silicone polymers and epoxy polymers. Epoxy polymers are characterized by the presence of a three-member cyclic ether group commonly referred to as an epoxy group, 1,2-epoxide, or oxirane. For example, diglycidyl ethers of bisphenol A may be used, in addition to compounds based on aromatic amine, triazine, and cycloaliphatic backbones. Another example includes the well-known Novolac polymers.

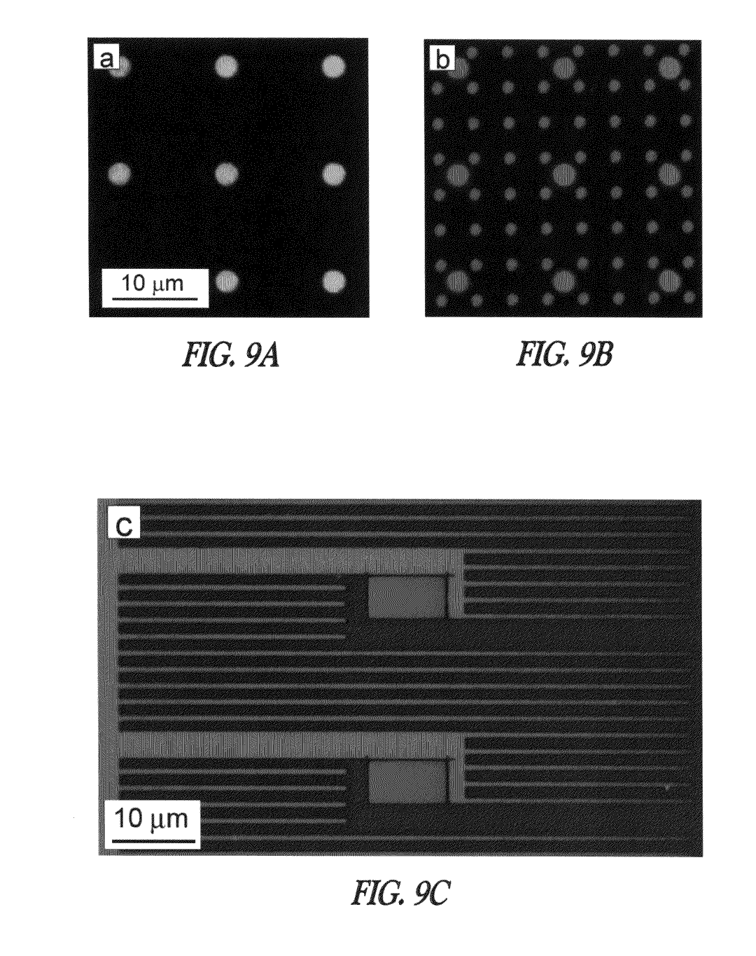

In some embodiments, microcontact printing can be used to generate patterns of signal molecules on the surfaces of substrates. The additive nature of this approach is well suited for this process. Combining several rounds of microcontact printing allows the patterning of either colocalized signal molecules (e.g., antibodies to both CD3 and CD28) in one predefined region of the substrate, or segregated signal molecules where each predefined region of the substrate contains one type of signal molecule. The overall pattern of signal molecules in predefined regions of the substrate captures the configuration of the cell-cell immunological synapse (see, e.g., FIG. 9).

For example, patterns of signal molecules can be generated by soft lithography procedures that involve spotting of signal molecules onto substrates using stamps. One type of stamp that can be used is a polydimethylsiloxane (PDMS) stamp. Pieces of polydimethylsiloxane (PDMS) that have been patterned can be used as stamps to generate patterns of signal molecules. In general, a patterned master is generated and the stamp is pressed against the master to form a relief pattern on the stamp. The PDMS stamp can be used in for microcontact printing on the substrate, or the stamp can attached to an external source by tubing so that liquid may be past through channels on its surface. In this second case it will often be laminated to a surface so that chemistry can be performed on that surface producing a pattern of the PDMS stamp on to the surface. Alternatively a PDMS stamp can be laminated to a second piece of PDMS to form a contained device. It is possible to pattern PDMS to generate micron-scale patterns.

When glass, silicon and/or metal substrates are employed, patterns of signal molecules can be generated on these surfaces by a variety of techniques. Patterning can involve formation of covalent or non-covalent bonds between the substrate and the signal molecules or linkers/binding materials on the substrate. For example, predefined regions of a glass or silicon substrate can be coated with metal (e.g., micro-dots of metal) and the signal molecules can be directly or, typically, indirectly attached to the metal. The non-metal parts of the substrate surface are generally passivated. The metal can be gold and/or palladium. The metal can be functionalized with thiols (e.g., derivatized alkanethiols) so that the thiol binds to the metal (gold) and the derivative on the alkane is available for binding to the signal molecule. In some embodiments, a gold-palladium (AuPd) mixture is used because it forms more robust films than pure Au, has a smaller grain size, making for flatter films, and is known to form stable linkage to thiol-containing molecules. Further details on how to make patterned surfaces are provided in Chemiavskaya et al. (J. Vac. Sci. Technol. B 23(6): 2972-78 (2005)).

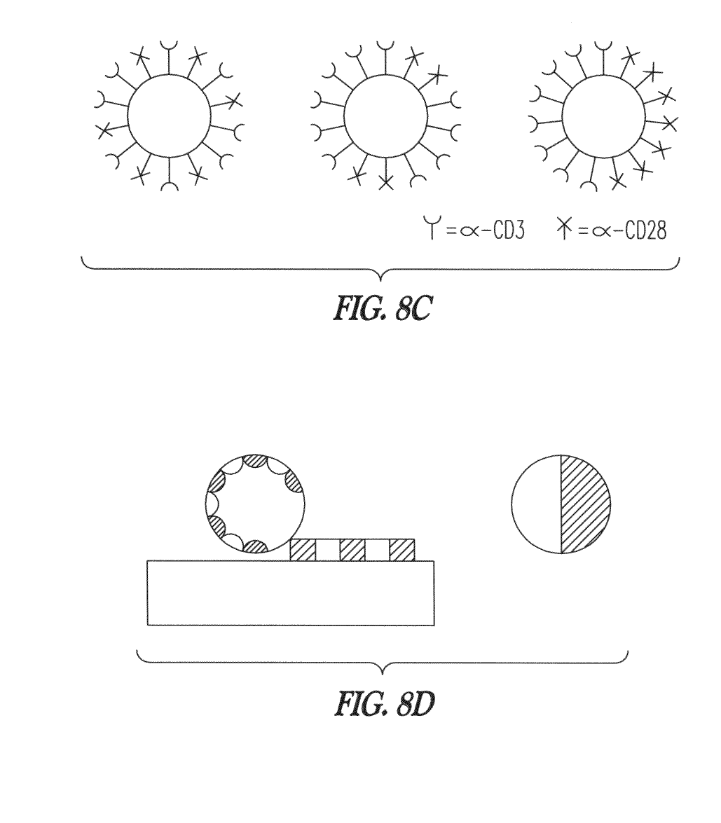

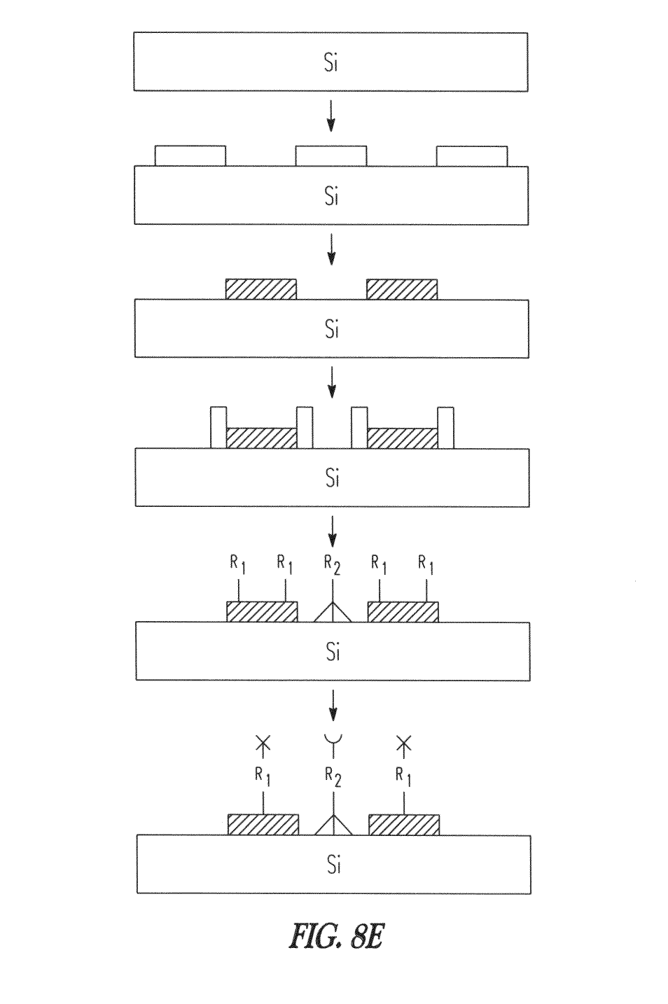

In other embodiments, porous multilayer structures can be generated (see, e.g., FIG. 8). Stacked layers of materials that are robust to processing can be used. The layers can comprise different materials, each with a ligand or an affinity for capturing a particular type of signal molecule. After layered the different materials, the layered substrate can be etched to generate pores, crevices, channels and other three-dimensional features. Different types of signal molecules can then added, where each type of signal molecule interacts or binds to a distinct layer of the substrate. These layered substrates therefore constitute porous structures that present signal molecules on the sides of the pores, crevices, channels and other etched features. More than one side of a cell may then interact with the signal molecules. Moreover, the cells can interact with the different signal molecules displayed by the different layers.

Another procedure for generating multifaceted substrates is entropic capturing. If the spatial distances between signal molecules (or predefined regions) of the substrate are of sufficiently different dimensions, entropic capturing can be used to pattern functionalized beads onto a topologically modified surface, as illustrated in FIG. 8B. Addition of polyethylene glycol polymer, or other large polymer or molecule, drives the assembly of beads into recesses in the substrate that are of an appropriate size. The topological surface of the substrate can be made rapidly using a variety of embossing, contact, or optical techniques.

Thus, signal molecules can be attached to substrates by a variety of procedures. In some embodiments an organic functionalized substrate can be used where functional groups on the substrate include amine, maleimide, carboxy, hydroxy, thiol or aldehyde moieties. Signal molecules can then be attached to the substrate by reaction with the functional groups at predefined regions of the substrate. Alternatively, the signal molecules can be covalently or non-covalently bound to proteins or epitopes that coat the substrate or predefined regions of the substrate. In some instances, layered materials are used on the substrate and, after etching, crevices or pores are formed that permit signal molecules and/or beads with signal molecules to occupy the crevices and/or pores. Antibodies and other binding partners can be placed on the substrates. Epitopes and tags can be incorporated into protein signal molecules (e.g., glutathione-S-transferase) that interact non-covalently with cognate ligands or proteins displayed on the substrate surface (such as glutathione).

In some embodiments, manufacturing the patterned substrate may also include use of a chemical block to prevent a further derivatization of the substrate surface with one or more signal molecules and/or to inhibit non-specific protein adsorption. The chemical block is generally a synthetic homopolymer or homo-oligomer. Examples include polyethylene glycol (PEG) and PEG analogs based on oligomeric N-substituted glycines or peptoids (e.g., other inert hydrophilic polymers) with termini that bind to the substrate. Treatment of the substrate surface with such a chemical block after spotting of the signal molecules onto the substrate may decrease non-specific protein binding to the array. In some cases, conventional blocking agents such as casein, bovine serum albumin and the like (protein blocks) may be used.

After application of the signal molecule to generate a pattern on the substrate, intracellular adhesion molecule (ICAM) can also be adsorbed to the open regions of the surface, providing an additional adhesive cue that can facilitate T cell activation.

### Methods of Manipulating T Cells

According to the invention, incubation of T cells on a pattern of selected signal molecules can modulate the expansion, selection and/or activation of the T cells. Therefore, a small population of syngeneic or autologous T cells can be obtained and that population can be manipulated by the present methods and/or devices to generate an expanded population of T cells, for example, an expanded population of memory or effector T cells.

The starting population of T cells can be a mixed population of cells, some of which are T cells. In some embodiments the starting population of cells preferably contains naïve T cells. The starting population of T cells can be obtained from bone marrow, thymus, circulating blood, lymph fluid and/or lymph nodes.

Thus, one aspect of the invention is a method of modulating T cells that includes incubating T cells on a substrate having pattern of signal molecules, where the pattern comprises a series of predefined regions on the substrate, each predefined region having at least one type of signal molecule, and wherein the predefined regions are spatially organized so as to optimize the modulation of the T cells. Thus, the spacing between the predefined regions with the signal molecules can be varied from about 0.1 to about 10 microns, or about 0.3 to about 7 microns, or about 0.5 to about 5 microns. In some embodiments, the predefined regions with signal molecules are optimally spaced about 1 to 2 microns apart.

For example, to activate the cells, the starting population (or sample) of T cells is incubated on a substrate with segregated predefined regions of anti-CD3 and anti-CD28 antibodies. In some embodiments, optimal IL-2 secretion occurs when the predefined regions are spaced about 1 to about 2 microns apart. Other signal molecules that may be used include B7, self peptides, major histocompatibility complex class I and/or II molecules, and/or antigenic peptides.

To expand the numbers of T cells the signal molecules can include CCL21 (SLC/6Ckine), interleukin-2, interleukin-12, anti-CD3 antibodies, anti-CD28 antibodies, self peptides, and MHC polypeptides. CCL21 is constitutively expressed by secondary lymphoid tissue and attracts CCR7-expressing mature dendritic cells and naïve T cells.

Other studies by the inventors indicate that naïve T cells respond with calcium spikes to spatially organized immobilized antibodies. In these studies, the anti-CD3/anti-CD28 patterned surfaces were additionally coated with ICAM-1 to provide the adhesive signal characteristic of an immunological synapse. TCR-positive cells (marker for T cells) form strong interference reflection microscopy (IRM) signals in contact with relatively large patterns containing both antibodies, when the patterns are squares measuring 5 μm on a side. On regions of the surface containing only small, sub-micrometer spots of these two antibodies, T cells were highly migratory but often formed strong IRM signals when in contact with these spots. Furthermore, T cells on these spots exhibited calcium spikes, indicative of recognition of both spots by T cells. These studies demonstrated that these micropatterned anti-CD3/anti-CD28 surfaces are effective at stimulating T cells and that T cells differentially recognize and respond to the antibody patterns based on the spatial organization of signal molecules.

The methods and devices of the invention enable enrichment of selected T cell types within T cell populations. Populations of T cells that are enriched for a particular phenotype can be administered to animals (e.g., human patients) as mixed, enriched populations of T cells. Alternatively, the mixed populations can be purified, for example, by cell sorting to enrich the T cell population even further.

The effectiveness of a pattern of signal molecules on a substrate can be assessed by evaluating whether the pattern generates an enriched population of the desired type, function or class of T cells.

The enriched population of T cells is enriched relative to the starting population of T cells (e.g., relative to the bone marrow, blood, lymph node, lymph fluid, or other source of T cells). The enriched population of T cells can also be enriched relative to a control T cell population that was generated by incubating a starting population of T cells (obtained from the same source as that used to generate the enriched T cell population) on a colocalized pattern of the same signal molecules used to generate the enriched population of T cells. According to the invention, segregated patterns of signal molecules optimally control and stimulate T cells.

In some embodiments, the enriched population of T cells can, for example, be an enriched population of memory or effector T cells.

### Administration

One aspect of the invention is a composition that includes a T cell population that has been manipulated or expanded using the methods and/or devices of the invention. T cells may be administered in any available procedure for introducing cells into a mammal. The cells may be introduced into a specific site in the body, for example, a site in need of T cells or a site that will facilitate further expansion, selection and/or activation of the T cells. However, in some embodiments, the T cells may find their way to diseased tissues, and/or to sites where further expansion, selection and/or activation of the T cells can occur, so local administration may not be needed.

T cells may be administered by intravascular, intravenous, intraarterial, intraperitoneal, intraventricular infusion, infusion catheter, balloon catheter, bolus injection, direct application to tissue surfaces during surgery, or other convenient routes. The cells can be washed after manipulation and/or cultured in an appropriate medium to insure their viability and to enhance their numbers. The cells can also be cultured in the presence of growth factors or interleukins. Prior to administration, the cells can be washed again, for example, in buffered physiological saline.

The volume of cells that is injected and the concentration of cells in the transplanted solution depend on the site of administration, the disease or disorder to be treated, and the species of the host. Preferably about one-tenth milliliter to about five milliliters is injected at a time. The number of cells injected can vary, for example, about 10² to about 10¹⁰ or about 10⁴ to about 10⁹ cells can be injected at one time. While a single injection may be sufficient, multiple injections may also be used.

T cell compositions suitable for injectable use often include a carrier. For compositions of T cells the carrier is generally a sterile aqueous solution. For example, the carrier can be a physiological saline solution or a buffered saline solution.

### Therapies

The T cells generated by methods of the invention and/or by using the devices of the invention are useful for replenishing depleted T cell populations and as therapeutic agents for treating diseases and disorders in an animal.

The term “animal,” as used herein, refers to an animal, such as a warm-blooded animal, which has an immune system deficiency or a condition or disease that depletes immune cells. Animals include cattle, buffalo, sheep, goats, pigs, horses, dogs, cats, rats, rabbits, mice, and humans. Also included are other livestock, domesticated animals and captive animals. The term “farm animals” includes chickens, turkeys, fish, and other farmed animals. Mammals and other animals including birds may be treated by the methods and compositions described and claimed herein.

In one embodiment, the T cells and/or compositions of the invention are used in a method for treating an animal with an immune cell deficiency that involves administering the T cells generated by the present methods and devices.

Mammals that are treated in this manner include those suffering from immune disorders. For example, the mammals may suffer from immunodeficiency viral infections (e.g., HIV-1, HIV-2, SIV), common variable immunodeficiency (CVI) disorder, primary immune deficiency diseases, severe combined immunodeficiency disease, and the like.

CVI is a group of rare genetic (primary) immunodeficiency disorders in which abnormalities in immune cell development (maturation) result in a decreased ability to appropriately produce antibodies in response to invading microorganisms, toxins, or other foreign substances. The symptoms of CVI usually become apparent during the second to the fourth decade of life. In some cases, individuals with Common Variable Immunodeficiency have an increased tendency to develop certain diseases characterized by abnormal tissue growths (neoplasms) that may be benign or malignant. In addition, some individuals with CVI may have an unusual susceptibility to certain autoimmune diseases. These disorders occur when the body's natural defenses against invading microorganisms mistakenly attack healthy tissue. The range and severity of symptoms and findings associated with CVI may vary from case to case.

Severe combined immunodeficiency disease is the most serious immunodeficiency disorder. It can be caused by several different genetic defects, most of which are hereditary. One form of the disorder is due to a deficiency of the enzyme adenosine deaminase. In the past, children with this disorder were kept in strict isolation, sometimes in a plastic tent, leading to the disorder being called “bubble boy syndrome.”

In another embodiment, the T cells are used for treating or preventing a pathologic state by stimulating an antigen-specific immune response against a particular antigen. The pathologic state can be characterized as any disease state experienced by a mammal, which can be treated or prevented using the methods of the present invention. For example, the T cells of the invention are useful for treating or preventing cancer, infectious diseases, and allergic conditions in a mammal.

When treating or preventing cancer in a mammal, a “cancer antigen” can be used to stimulate development of an enriched population of T cells. Such a cancer antigen is a molecule, such as a peptide, associated with a tumor or cancer cell surface and which is capable of provoking an immune response when expressed on the surface of an antigen presenting cell. Cancer antigens include a cancerous cell and immunogenic portions thereof.

The cancer can be lung cancer (e.g., small cell, non-small cell, Lewis lung cancer), bile duct cancer, breast cancer, bladder cancer, bone cancer, brain and spinal chord cancers, cervical cancer, colon cancer, endometrial cancer, esophageal cancer, gallbladder cancer, gastrointestinal cancer, laryngeal cancer, liver cancer, lymphoma, multiple myeloma, neuroblastoma, ovarian cancer, prostate cancer, pancreatic cancer, retinoblastoma, renal cancer, rectal cancer, skin cancer (e.g., melanoma and non-melanoma), stomach cancer, testicular cancer, thymus cancer, thyroid cancer, as well as other carcinomas and sarcomas. The cancer can be metastatic or non-metastatic.

The T cells of the invention are also useful for treating or preventing infectious diseases. An infectious disease, as used herein, is a disease arising from the presence of a foreign microorganism in the body. The T-cells are administered to augment humoral and/or cell-mediated response against an antigen of the microorganism. Peptide antigens can be used to generate T cells that specifically recognize those antigens on the foreign microorganism. Such peptide antigens are well-known and used routinely in the alt.

The infectious disease is a viral, bacterial or fungal infection, where cell-mediated immune responses are needed. The infectious virus can be any virus known in the art including both RNA and DNA viruses. For example, the virus can belong to the family of Retroviridae (e.g., human immunodeficiency viruses, such as HIV-1); Picornaviridae (e.g., aphthoviruses, cardioviruses, enteroviruses, hepatoviruses, parechoviruses, rhinoviruses); Togaviridae (e.g., alphaviruses, rubiviruses); Rhabdoviridae (e.g., cytorhabdoviruses, ephemeroviruses, lyssaviruses, vesiculoviruses); Filoviridae (e.g., ebola viruses); Paramyxoviridae (e.g., morbilliviruses, paramyxoviruses, rubulaviruses, pneumoviruses); Orthomyxoviridae (e.g., influenza viruses); Arenaviridae (hemorrhagic fever viruses); Reoviridae (e.g., aquareoviruses, orbiviruses, rotaviruses); Bimaviridae; Hepadnaviridae (Hepatitis B virus); Papillomaviridae (papilloma viruses, polyoma viruses); Adenoviridae; Herpesviridae (herpes simplex virus (HSV) 1 and 2, varicelloviruses, cytomegalovirus (CMV), herpes virus); Poxyiridae (variola viruses, vaccinia viruses, pox viruses); Iridoviridae (e.g., iridoviruses, ranaviruses); and unclassified viruses (e.g., the etiological agents of Spongiform encephalopathies; Hepatitis C; Norwalk and related viruses; and astroviruses).

The infectious fungus also can be any fungus known in the art. Examples of infectious fungi can include, for example, Histoplasma capsulatum, Coccidioides immitis, Blastomyces dermatitidis, Chlamydia trachomatis, and Candida albicans. Other infectious organisms include: Plasmodium such as Plasmodium falciparum, Plasmodium malariae, Plasmodium ovale, and Plasmodium vivax and Toxoplasma gondii.

The infectious disease also can be a bacterial infection. Both gram-negative (e.g., Escherichia coli (E. coli), Pseudomonas species, and Salmonella species) and gram-positive bacteria (e.g., Pasteurella species, Staphylococcus species, and Streptococcus species) can serve as antigens in a mammal. Specific examples of infectious bacteria include, but are not limited to, Helicobacter pylori, Legionella pneumophila, Staphylococcus aureus, Neisseria gonorrhoea, Neisseria meningitidis, Listeria monocytogenes, Streptococcus pyogenes, Streptococcus faecalis, Streptococcus bovis, Streptococcus pneunmoniae, Haemophilus influenzae, Bacillus anthracis, Corynebacterium diphtheriae, Erysipelothrix rhusiopathiae, Clostridium perfringens, Clostridium tetani, Enterobacter aerogenes, Klebsiella pneunmoniae, Fusobacterium nucleatum, Streptocabillus moniliformis, Treponema palladium, and Actinomyces israelii, as well as various species belonging to the genera of Mycobacterium, Campylobacter, Enterococcus, Bacteroides, and Leptosipira.

The T cells of the invention are also useful for treating or preventing allergic conditions. An allergic condition can occur when a mammal acquires hypersensitivity to an allergen. An “allergen” refers to a substance that can induce an allergic response in a susceptible mammal. The allergen is typically an animal or plant allergen such as, for example, pollens, insect venoms, animal dander, dust, and fungal spores, but also can be a food or drug (e.g., penicillin). Allergic conditions include, but are not limited to, eczema, allergic rhinitis or coryza, hay fever, bronchial asthma, urticaria (hives) and food allergies, and other atopic conditions. The method is accomplished in the same way as described above for cancer and infectious disease, except that the immune response is elicited towards an antigen that is specific for an allergen.

Whether the T cells of the invention are used to treat or prevent a pathologic state in a mammal will depend upon the condition of the pathologic state (e.g., whether or not the mammal has developed the pathologic state). If the pathologic state or other condition associated with the pathologic state is present, the methods of the invention can be used to treat the pathologic state. Alternatively, the mammal may have not yet acquired the pathologic state but may be at risk of developing the pathologic state. A mammal at risk of developing a pathologic state is one who is or who has a high probability of developing the pathologic state. These mammals include, for example, mammals having a genetic abnormality, the presence of which has been demonstrated to have a correlative relation to a higher likelihood of developing a pathologic state, and mammals exposed to harmful agents, such as chemical toxins (e.g., tobacco, asbestos) or radiation.

The following Example illustrate the invention but are not limiting thereof.

### EXAMPLE 1

### Materials and Methods

This Example describes certain materials and methods that have been useful in the development of the invention.

Substrate preparation. Glass coverslips were cleaned by immersion into hot detergent (Linbro 7×, diluted 1:3 with deionized water), rinsed extensively with deionized water, and then baked at 450° C. for 6 hours. For microcontact printing, topological masters were made by ebeam lithography using a 1 μm polymethyl methacrylate (PMMA) layer, spin-coated onto silicon wafers.

Hybrid, dual rigidity stamps were made by spin-coating a thin layer of hard polydimethylsiloxane (PDMS) onto the masters (see, Odom et al., (2002) Langmuir 18, 5314-5320; Schmid, H. & Michel, B. (2000) Macromolecules 33, 3042-3049). A thick layer of Sylgard 184 (Dow Corning) was then added. Stamps were used in this as-cast, hydrophobic state for patterning.

Stamps were coated with antibodies in the indicated patterns (maintaining a total antibody concentration of roughly 25 μg/ml) for 30 minutes, rinsed with PBS, PBS+0.05% Tween-20 and deionized water, then placed in contact with the cleaned coverslips for 1 minute. Substrates were rinsed extensively with PBS, and then coated with ICAM-1 (R&D Systems) at a concentration of 2 μg/ml for 2 hours. For visualization purposes, a fraction of the TS2/4 and CD28 antibodies was labeled using Alexa Fluor 568 (Invitrogen). For estimation of surface concentration, antibodies were labeled with Texas Red, mixed (in defined ratios) with unlabeled antibodies (to reduce self-quenching), and patterned by microcontact printing. The dye/protein ratio was targeted to be 4:1 and measured by comparing absorbance at 280 nm and 595 nm. Supported lipid bilayers were formed on separate substrates by fusion of vesicles of egg phosphatidylcholine (Avanti Polar Lipids) supplemented with specific concentrations (0.03-0.14 mol %) of Texas Red-DHPE (Invitrogen) and prepared by extrusion (Kung et al., (2000) Langmuir 16, 6773-6776). These well-defined structures provide a highly consistent areal concentration of lipids, which was assumed to be about 3.3×10⁶ lipids per square micron for a bilayer of egg phosphatidylcholine.

T cell assays. Freshly isolated CD4+ T cells were purified from lymph nodes of C57BL/6 mice by negative selection (Dynal CD4 Negative Isolation Kit, Invitrogen). Naïve T cells comprised typically greater than 85% of the cell population, as quantified by immunofluorescent detection of cells exhibiting a combination of high/low levels of CD44/CD62L respectively. For experiments, cells were resuspended in RPMI-1640 (Invitrogen)+5% mouse serum (eBioscience) and seeded onto the micropatterned substrates at a density of 2×10⁴ cells/mm².

Immunochemistry. PKCθ localization was determined using a polyclonal antibody to the C-terminus of this protein (Santa Cruz Biotechnology). Cells were fixed and extracted using 4% paraformaldehyde+0.25% Triton X-100 (4° C.) and then stained using standard immunofluorescence techniques. Nuclear translocation of NF-κB was measured using a polyclonal antibody to the p65 subunit (Santa Cruz Biotechnology). Cells were fixed and extracted, stained for NF-κB, and counterstained using the SYTOX Green DNA dye (Invitrogen). A region representing the core of each nucleus was isolated from the central third of each nucleus, identified in image stacks of cell nuclei (DNA), and the average NF-κB staining in these regions (FIG. 4A) was compared across the different surfaces. Regions adjacent to, but not including, adherent cells were used to determine a background signal.

IL-2 assays. IL-2 secretion was compared using a cell surface-capture assay kit (Miltenyi Biotec). Immediately after purification, cells were incubated with an IL-2 capture reagent then seeded onto substrates. After one hour, the samples were gently rinsed with warm (37° C.) RPMI-1640 medium to remove unattached cells. After six hours (total) incubation (37° C.), cells were rinsed and incubated with a fluorescently-labeled antibody to IL-2. The average fluorescence intensity associated with APC-labeled IL-2 was estimated by microscopy on a cell by cell basis. Experiments of Akt inhibition were carried out by including triciribine (Calbiochem) in the cell culture media at a concentration of 10 μM.

Visualization of dendritic cell-T cell immune synapses. BAC Transgenic mice expressing full-length CD80 appended with Enhanced Cyan Fluorescent Protein (eCFP) were generated using established methods (Tseng, S.-Y., Liu, M., & Dustin, M. L. (2005) J Immunol 175, 7829-7836; Sparwasser et al. (2004) Genesis 38, 39-50). The genetic background of the mice employed was CD80/CD86−/− B6. The distribution of CD80-eCFP in dendritic cells from these animals was identical to that of wild-type CD80 in cells from B6 mice.

Dendritic cells were purified from spleens of these mice using CD11c beads (Miltenyi), and the purified dendritic cells were activated with 12.5 μg/ml of lipopolysaccharide (LPS; Sigma) and 1 μg/ml of anti-CD40 (BD Pharmingen) for 16-18 hrs. Naïve CD4+ T cells were purified from spleens of OTII×Rag^{-/-} mice by negative selection (Miltenyi). Activated cells were generated by treating these cells with 5 μM of OVA peptide (OVA323-339, ISQAVHAAHAEINEAGR, SEQ ID NO:1) in RPMI1640 (Invitrogen) supplemented with 10% fetal bovine serum, L-glutamate, non-essential amino acids, sodium pyruvate, and 2 μM 2-mercaptoethanol. Cells were used on day 5-8. Activated dendritic cells were plated onto glass-bottom chambers (coated for 1 hr. with 200 μg/ml human fibronectin) and then loaded with OVA peptide (SEQ ID NO:1). OTII T cells were then added to these chambers. After 30 minutes, these cells were fixed and immunostained for T-cell receptor (TCR) using H57-Alexa568 antibody.

Image quantification and statistics. Processing of microscopy data was carried out using the MetaMorph (Molecular Devices) software package. Enface projections of dendritic cell-T cell interfaces were generated from confocal stacks using the Volocity (Improvision) software package. Statistical analysis of IL-2 secretion, which exhibited a non-normal distribution, was carried out using Kruskal-Wallis (KW) approaches. Significance levels of α=0.05 and 0.01 (as a more stringent test when the null hypothesis could be rejected at α=0.05) were used as indicated in the text. When presented as box plots, the whiskers and elements of the boxes corresponding to 5, 25, 50, 75, and 95 percentiles. All samples from with a single experiment were processed and imaged in one session, and statistical comparisons were made only within each experiment. Experiments were carried out at least three times to establish consistency.

### EXAMPLE 2

### T Cells Recognize and Respond to Micrometer-Scale Organization in the Presentation of TCR/CD28 Ligands

This Example shows that the spatial organization of signal molecules presented to T cells modulates the response of those T cells.

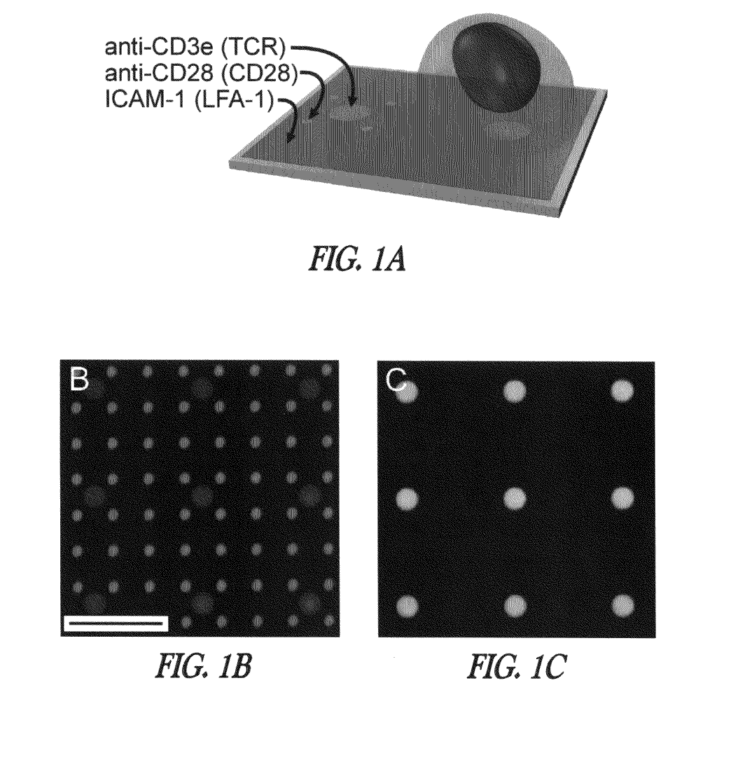

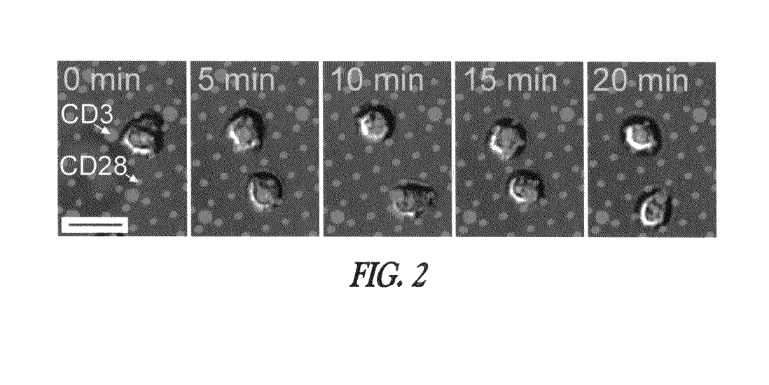

Patterning of multicomponent surfaces. Experiments were designed to create trifunctionalized surfaces containing independent patterns of activating antibodies to CD3 and CD28, surrounded by ICAM-1. Specifically, “segregated” patterns were generated consisting of anti-CD3 circles surrounded by satellite features of anti-CD28 as illustrated in FIG. 1. Combining rounds of microcontact printing, one for each of the two different antibodies, provided an effective means for creating sharply defined surfaces with minimal cross-contamination between regions (FIG. 1B). Colocalized patterns of anti-CD3 and anti-CD28 (FIG. 1C) were defined by combining these antibodies together in solution for use in a single microcontact printing step.

T cell activation is strongly influenced by the surface density of ligands (signal molecules) presented to the cell. To control this factor, the surface density of antibodies deposited by microcontact printing was measured using a microscopy-based approach (see Example 1). The printing conditions employed yielded an antibody density of approximately 200 molecules/μm² with a variation of 15% across a substrate and between samples. This concentration represented a small fraction (less than 10%) of a close-packed monolayer of protein, but is in the range required to evoke a costimulatory response and much larger than that needed for effective TCR engagement (Bromley et al. (2001) Nat Immunol; 2, 1159-1166).

To adjust the surface density of a specific antibody, it was mixed with an inert antibody in the stamping solution while keeping the total concentration of protein constant. Separate experiments verified that the surface density of the anti-CD3 and anti-CD28 antibodies changed linearly upon mixing with TS2/4 antibody. Each surface was then coated with ICAM-1, filling in the regions separating and surrounding the antibody patterns.

T cell recognition of costimulatory arrays. CD4+ T cells (>85% naïve cells) were isolated from mouse lymph nodes and seeded onto patterned surfaces. As illustrated in FIG. 2, cells migrated along the patterned surfaces, largely through interactions with the integrin ligand ICAM-1; few cells were able to attach to surfaces containing anti-CD3 and/or anti-CD28 alone. Cell migration halted when cells encountered a feature of anti-CD3, typically within the first 30 minutes of being seeded onto a substrate, recapitulating the “stop” signal associated with TCR engagement by primed T cells observed by Doh and Irvine (Proc Nat Acad Sci, USA 103, 5700-5705 (2006)). On the arrays described herein, the cells remained highly active, extending processes away from the anti-CD3 dot but not releasing this feature over 2 hours. Cells were able to recognize anti-CD3 features as small as 1 μm in diameter and patterned using 1:10 (w/w) mix of anti-CD3: TS2/4, corresponding to a density of about 20 anti-CD3 molecules/μm², the smallest size and lowest concentration that was examined. In contrast to anti-CD3, cell migration was not influenced by regions of anti-CD28 antibody alone, even when patterned at their highest surface concentration (i.e., not diluted with TS2/4). Cells continued to explore these surfaces until encountering an anti-CD3 feature, supporting an instructive, rather than adhesive, role of CD28 in T cell co-stimulation. Based on these observations, a set of three standard patterns were established:
CD3-only (CD3): a square array of 2 μm-diameter dots patterned using a 1:10 mix of anti-CD3: TS2/4 (2.5 μg/ml: 25 μg/ml) and spaced at 12 μm pitch.
Colocalized (COL): a square array of 2 μm-diameter dots patterned using a 1:10 mix of anti-CD3: anti-CD28 spaced at a 12 μm pitch. Costimulatory molecules are thus presented together with the TCR ligands, typically in the central region of the immune synapse (IS).
Segregated (SEG): a square array of 2 μm-diameter dots (12 μm center-to-center pitch), patterned using a 2.5 μg/ml:25 μg/ml mix of anti-CD3:TS2/4. This was overlaid with an array of 1 μm diameter dots of anti-CD28 (25 μg/ml) spaced at a 4 μm pitch, which promotes engagement of CD28 in the IS peripheral region. A naïve T cell of roughly 7-μm diameter and centered on the anti-CD3 spot will thus be exposed to approximately equal amounts of anti-CD28 signal on the colocalized and segregated surfaces.

Separate experiments indicated that cell response to anti-CD28 was independent of coating concentration when this antibody was included at over half mass fraction of the total protein (i.e., a solution containing a 1:1 mix of anti-CD28 and TS2/4). For simplicity, the anti-CD28 antibody was used at high surface concentration throughout this study. Patterned antibodies were stable throughout the duration of these experiments, and in fact can last up to weeks.

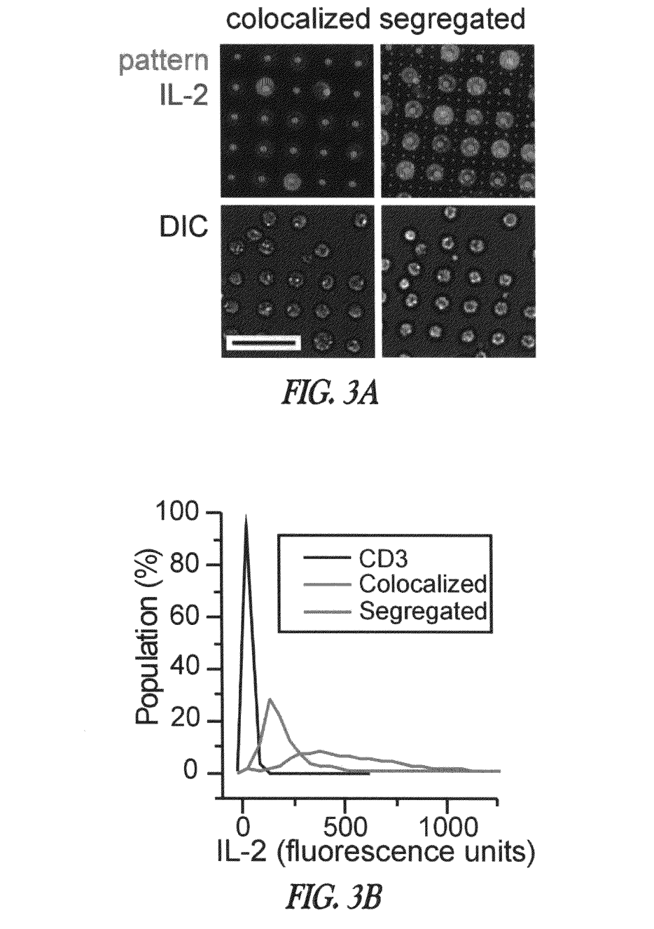

Costimulation array geometry modulates IL-2 production. IL-2 secretion by naïve T cells over a period of 6 hours was measured using a surface-capture technique, in which IL-2 is captured on the cell from which it is released and detected using a second, fluorescently labeled antibody. Thus, such a technique provides a cell-by-cell, spatially resolved measure of IL-2 secretion (FIG. 3A). As illustrated in FIG. 3A and the histograms in FIG. 3B, IL-2 secretion was higher (2-to-3 fold increase in median values) on surfaces containing segregated, compared to co-localized, patterns of anti-CD3 and anti-CD28 antibodies. Thus, spatial patterns containing separate dots of anti-CD3 and anti-CD28 antibodies cause T cells to secrete more IL-2 than when the anti-CD3 and anti-CD28 antibodies are combined together and presented to T-cells. Moreover, IL-2 secretion on CD3-only surfaces was dramatically lower than observed for surfaces with both anti-CD3 and anti-CD28 antibodies. In particular, IL-2 secretion was lower by a factor of 15 compared to that observed with the colocalized anti-CD3 and anti-CD28 antibody pattern. The three conditions tested yielded results that are statistically different from each other, as determined by both ANOVA and Kruskall-Wallis (KW) methods (α=0.01, n>1000 cells per surface).

These results demonstrate that more than one signal is needed for optimal activation of naïve T cell preparations, and that spatially separated costimulatory signals provide T cell optimal activation.

Interestingly, alignment between the anti-CD3 and anti-CD28 features on the segregated surfaces had only a minor effect on IL-2 secretion. The surfaces employed here exhibit a gradual phasing in which one region is patterned with well-separated anti-CD3 and anti-CD28 features, such as that shown in FIG. 1B, while at some distance away (typically hundreds of micrometers), individual anti-CD28 dots overlapped with a larger anti-CD3 feature. While alignment of microcontact printing steps is possible, the small dimensions of these surfaces together with the elastic nature of the stamps made precise registration of the two antibody patterns difficult. However, the microscopy based approach used to compare IL-2 secretion allowed correlation with the geometry of anti-CD3 and anti-CD28 ligands for each cell. Data from segregated surfaces were divided into three groups: cells on well-separated patterns of a central anti-CD3 feature equidistant from four anti-CD28 dots; overlapping patterns in which an anti-CD3 feature completely encompassed a smaller anti-CD28 dot; and the remainder of intermediate alignment. In each experiment, IL-2 secretion was approximately 10% higher on well-aligned patterns compared to overlapping patterns.

When analyzed using ANOVA and KW methods (α=0.05, n=700-3000 cells per group) statistical significance of this difference was often, but not unanimously, indicated. This lack of consistency might relate to the statistical power of each tests, given the limited number of cells available on some surfaces and that significant differences were seen on surfaces offering the greatest (n about 3000 for each group) number of cells. Given that statistical significance was not consistently detected and that the 10% difference between well-separated and overlapping patterns is small compared to that between colocalized and segregated surfaces we consider the effect of pattern alignment to be minor, and include cells from all three groups in data of the segregated surfaces.

NF-κB translocation. A major checkpoint in the pathway to IL-2 secretion is activation of NF-κB, involving translocation from cytosolic stores to the cell nucleus where it promotes transcription of IL-2 mRNA. However, Sanchez-Lockhart et al. (20, 21) identify an alternative route to IL-2 secretion that involves stabilization of IL-2 mRNA, rather than increased transcription, and is associated with trans costimulation, in which TCR and CD28 ligands are presented at different faces of the T cell. To determine whether these results are related to signaling within the immune synapse or a manifestation of trans initiated mRNA stabilization, we compared nuclear translocation of NF-κB. T cells were fixed 4 hours after seeding and stained for NF-κB (FIG. 4A). The central region of each cell nucleus was segmented and average intensity of NF-κB staining compared between patterned surfaces. NF-κB translocation was different on each pattern (α=0.01, n>80 on each surface) and followed that of IL-2 secretion (FIG. 4B), with highest levels observed on the segregated pattern, followed by the colocalized and, finally, the CD3-only surfaces. The ability of T cells to discriminate between the segregated and colocalized patterns is driven, at least in part, by processes upstream of NF-κB translocation.

PKCθ and Akt. Experiments were then designed to assess PKCθ and Akt signaling, both of which lead to NF-κB translocation. Engagement of TCR and CD28 serves to recruit PKCθ to the cell membrane, an important step for subsequent activation of this kinase (Schmitz, M. L. & Krappmann, D. (2006) Cell Death Differ 13, 834-842; Weil, R. & Israel, A. (2006) Cell Death Differ 13, 826; Altman, A. & Villalba, M. (2003) Immunol Rev 192, 53-63; Bi et al. (2001) Nat Immunol 2, 556; Sims et al. (2007) Cell 129, 773-785; Sun et al., (2000) Nature 404, 402-407). Immunochemical staining for PKCθ in cells fixed at time points from 30 minutes to 4 hours revealed a similar localization of this kinase on all surfaces, predominantly with areas overlying the anti-CD3 antibodies (FIG. 5). A minor, diffuse distribution of PKCθ in proximal areas surrounding the anti-CD3 features was also observed, which was slightly expanded on the segregated surfaces in areas between anti-CD3 and anti-CD28 regions (FIG. 5, bottom row). A small number of PKCθ clusters were observed on features of anti-CD28 (without anti-CD3), but were less prominent than those on the anti-CD3 circles, and many anti-CD28 features showed no colocalization with PKCθ. This observation was surprising, as Tseng et al. observed that PKCθ in naïve cells interacting with artificial APCs colocalizes with CD28 and not TCR (J Immunol 175, 7829-7836 (2005)). In addition, both the Tseng study and the more recent study by Sims et al., using supported lipid bilayers to replace the antigen presenting cells (APC), observed that PKCθ is localized to the pSMAC or the borders between this region and the cSMAC. However, other studies indicate that cSMAC localization of PKCθ as necessary for full T cell activation. Notably, ligands to both TCR and CD28 are present throughout the cell interface in many of these studies. By completely separating the anti-CD3 and anti-CD28 signals, the results described herein indicate that low, background levels of TCR engagement may be required to promote the PKCθ localization with CD28 observed in such studies. Alternatively, sites of CD28 engagement promote PKCθ binding to the membrane, but these clusters are not fixed in place, and instead migrate to the cSMAC region. The similarity in distribution on the colocalized and segregated surfaces suggests that PKCθ may not be a major mechanism by which these cells distinguish between the two patterns. It is noted that a similar distribution of PKCθ was observed on the CD3-only surface. However, cells withdrew from the CD3-only surfaces over several hours (as illustrated by the cell outlines in FIG. 5) but not the colocalized or segregated surfaces, reinforcing the importance of CD28 engagement in modulating cell function.

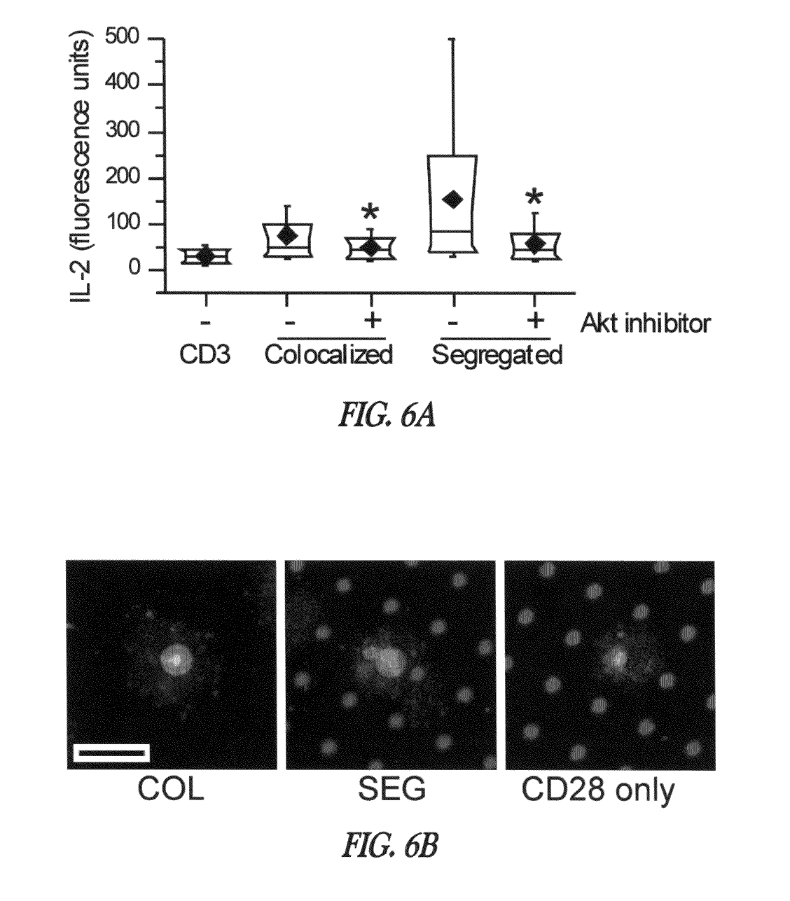

Akt activity, like PKCθ, is dependent on association of this protein with the cell membrane but also involves release of phosphorylated Akt to allow interaction with downstream molecules in the cytosol and other cellular structures. Immunochemical staining would thus provide only a limited view into Akt activity. Instead, a pharmacological approach was used to examine the role of this pathway, carrying out the IL-2 secretion assay in the presence of triciribene, an Akt inhibitor that does not affect upstream molecules such as PI3K and PDK (which are involved with other pathways, including PKCθ activation) (Karst et al. (2006) Cancer Res 66, 9221-9226; Yang et al. (2004) Cancer Res 64, 4394-4399). Inclusion of this inhibitor reduced IL-2 secretion on both colocalized and segregated patterns (FIG. 6). Interestingly, these reduced levels were similar to each other (no difference observed at α=0.05) indicating that the ability of T cells to differentially recognize the colocalized and segregated surfaces can be completely accounted for by the activity of Akt.

Like many elements of T cell signaling, CD28 and Akt interact not directly but through the activity of other proteins. In this case, PI3K (which does interact with CD28) is believed to facilitate this interaction by converting the membrane component PIP2 to PIP3, to which Akt binds through PH domains, however similar activities are also involved in the PKCθ pathway (Okkenhaug et al. (2004) Biochem Soc Trans 32, 332-335; Parry et al. (2007) Trends Immunol 28, 161-168). Changing the distribution of active CD28 complexes may thus change the extent of PIP3-rich regions of the lipid membrane. It is notable in this regard that PIP3 is observed to have a widespread distribution in T-B conjugates in which peripheral TCR microclusters are likely to sustain signaling (Costello et al. (2002) Nat Immunol 3, 1082-1089). On the surfaces employed herein, cells on colocalized patterns exhibited small, bright clusters of PI3K over the anti-CD3/CD28 features when examined 10 minutes after seeding. On segregated patterns, these clusters were also localized over the central anti-CD3 feature, with minimal association with the anti-CD28 dots, suggesting the surprising result that PI3K is not involved in modulating Akt signaling as a function of pattern geometry. Notably, cells on regions of the segregated surfaces that lacked anti-CD3 expressed PI3K clusters that associated with the anti-CD28 dots (FIG. 6B), suggesting a more complex interaction between CD3 and CD28 pathways. The full extent of this interaction, as well as the mechanism for modulating Akt signaling as a function of ligand geometry, remains unclear.

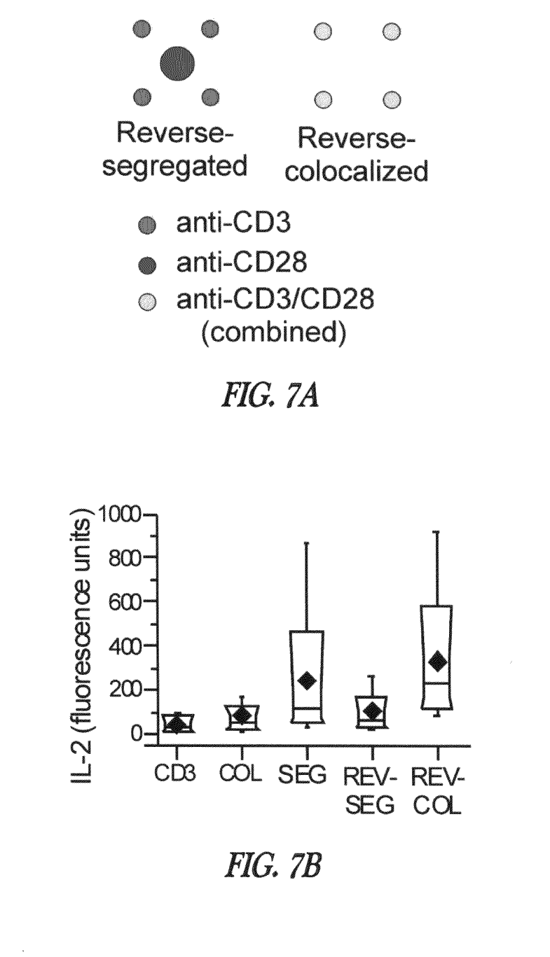

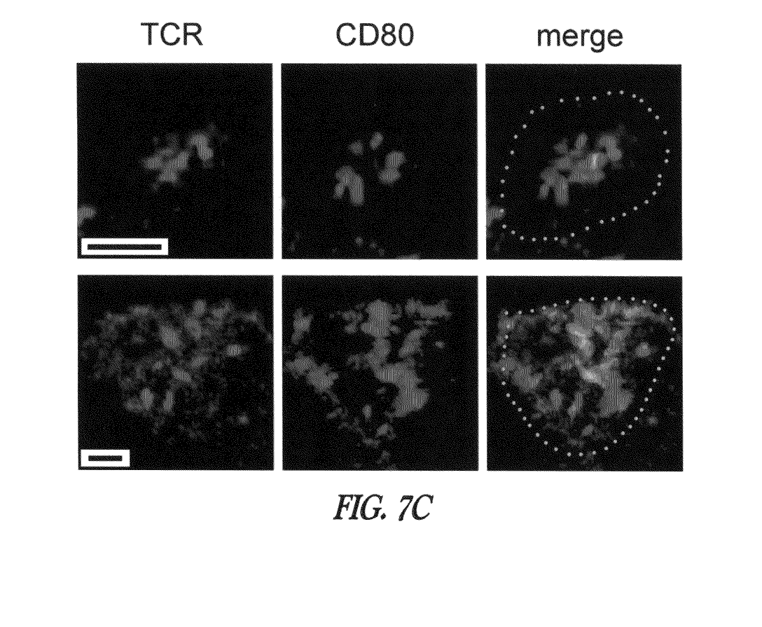

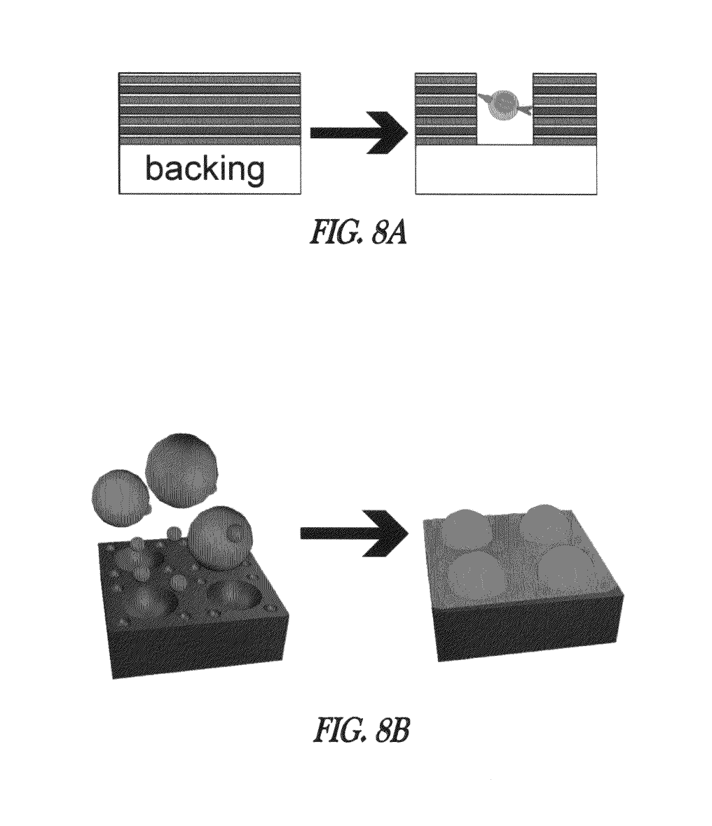

IL-2 secretion correlates primarily with CD28 geometry. Two variations on the “standard” patterns of TCR/CD28 ligands were examined. The first, a “reverse segregated” pattern, consists of a central 2 μm feature of anti-CD28, surrounded by 1 μm dots of anti-CD3 (FIG. 7A). Cells preferentially attached to the anti-CD3 dots, typically interacting with three, but sometimes four, individual features. IL-2 secretion was significantly decreased compared to the (standard) segregated pattern, but higher than on the (standard) colocalized surface (α=0.01); cells that were not in contact with a feature of anti-CD28 were excluded from this analysis. The last pattern was a “reversed colocalized” configuration (FIG. 7A), consisting of an array of 1 μm dots, spaced at 4 μm intervals and containing both anti-CD3 and anti-CD28. As shown in FIG. 7B, IL-2 secretion by cells on this final surface was even higher than that observed on the (standard) segregated pattern (α=0.05).

The geometry of CD28 engagement within the immune synapse may thus be the primary regulator of IL-2 secretion. That is, a set of four distributed 1 μm dots of anti-CD28 promotes higher IL-2 secretion than a single 2 μm feature. IL-2 secretion was highest on the reverse colocalized pattern, suggesting that distributing the TCR signaling, when accompanied by effective CD28 signaling, also enhances cell activation. Indeed, earlier reports with previously activated T cells support the idea that peripheral engagement of TCR leads to enhanced function (Mossman et al. (2005) Science 310, 1191-1193). In support of this idea, the immune synapse formed between T cells and dendritic cells is characterized by a multi-focal distribution of TCR complexes (Brossard et al. (2005) Eur J Immunol 35, 1741-1753).

To examine the localization of CD80 within these synapses, CD4+ T cells from OTII transgenic mice were seeded onto dendritic cells loaded with OVA peptide and expressing an eCFP-tagged CD80. For both naïve and previously activated T cells (FIG. 7C), CD80 clusters were multifocal, separated by micrometer scale distances (particularly in previously activated cells), and distributed across the immune synapse. Furthermore, these clusters were segregated from TCR clusters. Thus, the pattern geometries examined here are observed in native cell-cell interfaces. Dendritic cells, as highly effective APCs, may utilize both distribution and segregation of TCR and CD28 signals to drive T cell function.

In summary the results provided herein demonstrate that the micro-scale organization of TCR and CD28 ligands within the immune synapse modulates T cell activation, an important process in coordinating the immune system. Moreover, the increase in IL-2 secretion induced by localizing the CD28 signal to the periphery of the immune synapse is not simply the result of amplification of a single, unifying costimulation pathway, rather it is the selective enhancement of specific pathways (namely, the Akt pathway) that are otherwise acting in parallel. Changing the balance of signaling pathways may have significant impacts on the T cell classes that subsequently result from this process. These results further demonstrate the role of spatial organization of ligands as part of the language of cell-cell communication.

### REFERENCES

1. Bromley, S. K., Burack, W. R., Johnson, K. G., Somersalo, K., Sims, T. N., Sumen, C., Davis, M. M., Shaw, A. S., Allen, P. M., & Dustin, M. L. (2001) Annu Rev Immunol 19, 375-396.
2. Davis, D. M. (2006) Sci Am 294, 48-56.
3. Davis, M. M., Krogsgaard, M., Huppa, J. B., Sumen, C., Purbhoo, M. A., Irvine, D. J., Wu, L. C., & Ehrlich, L. (2003) Annu Rev Biochem 72, 717-742.
4. Groves, J. T. & Dustin, M. L. (2003) J Immunol Meth 278, 19-32.
5. Krummel, M. F. & Macara, I. (2006) Nat Immunol 7, 1143-1149.
6. Kupfer, A. (2006) Immunity 25, 11-13.
7. Tseng, S.-Y., Liu, M., & Dustin, M. L. (2005) J Immunol 175, 7829-7836.
8. Doty, R. T. & Clark, E. A. (1996) J Immunol 157, 3270-3279.
9. Bromley, S. K., laboni, A., Davis, S. J., Whitty, A., Green, J. M., Shaw, A. S., Weiss, A., & Dustin, M. L. (2001) Nat Immunol; 2, 1159-1166.
10. Grakoui, A., Bromley, S. K., Sumen, C., Davis, M. M., Shaw, A. S., Allen, P. M., & Dustin, M. L. (1999) Science 285, 221-227.
11. Mossman, K. D., Campi, G., Groves, J. T., & Dustin, M. L. (2005) Science 310, 1191-1193.
12. Doh, J. & Irvine, D. J. (2006) Proc Nat Acad Sci, USA 103, 5700-5705.
13. Whitesides, G. M., Ostuni, E., Takayama, S., Jiang, X., & Ingber, D. E. (2001) Annu Rev Biomed Eng 3, 335-373.
14. Chen, C. S., Mrksich, M., Huang, S., Whitesides, G. M., & Ingber, D. E. (1998) Biotechnol Progress 14, 356-363.
15. Kumar, A. & Whitesides, G. M. (1993) Appl Phys Lett 63, 4.
16. Schmitz, M. L. & Krappmann, D. (2006) Cell Death Differ 13, 834-842.
17. Weil, R. & Israel, A. (2006) Cell Death Differ 13, 826.
18. Shi, P., Shen, K., & Kam, L. (2007) Dev Neurobiol in press.
19. James, C. D., Davis, R., Meyer, M., Turner, A., Turner, S., Withers, G., Kam, L., Banker, G., Craighead, H., Isaacson, M., et al. (2000) IEEE Tras Biomed Eng 47, 17-21.
20. Sanchez-Lockhart, M., Marin, E., Graf, B., Abe, R., Harada, Y., Sedwick, C. E., & Miller, J. (2004) J Immunol 173, 7120-7124.
21. Sanchez-Lockhart, M. & Miller, J. (2006) J Immunol 176, 4778-4784.
22. Altman, A. & Villalba, M. (2003) Immunol Rev 192, 53-63.
23. Bi, K., Tanaka, Y., Coudronniere, N., Sugie, K., Hong, S., van Stipdonk, M. J. B., & Altman, A. (2001) Nat Immunol 2, 556.
24. Sims, T. N., Soos, T. J., Xenias, H. S., Dubin-Thaler, B., Hofman, J. M., Waite, J. C., Cameron, T. O., Thomas, V. K., Varma, R., Wiggins, C. H., et al. (2007) Cell 129, 773-785.
25. Sun, Z., Arendt, C. W., Ellmeier, W., Schaeffer, E. M., Sunshine, M. J., Gandhi, L., Annes, J., Petrzilka, D., Kupfer, A., Schwartzberg, P. L., et al. (2000) Nature 404, 402-407.
26. Karst, A. M., Dai, D. L., Cheng, J. Q., & Li, G. (2006) Cancer Res 66, 9221-9226.
27. Yang, L., Dan, H. C., Sun, M., Liu, Q., Sun, X.-m., Feldman, R. I., Hamilton, A. D., Polokoff, M., Nicosia, S. V., Herlyn, M., et al. (2004) Cancer Res 64, 4394-4399.
28. Okkenhaug, K., Bilancio, A., Emery, J. L., & Vanhaesebroeck, B. (2004) Biochem Soc Trans 32, 332-335.
29. Parry, R. V., Riley, J. L., & Ward, S. G. (2007) Trends Immunol 28, 161-168.
30. Costello, P. S., Gallagher, M., & Carntrell, D. A. (2002) Nat Immunol 3, 1082-1089.
31. Brossard, C., Feuillet, V., Schmitt, A., Randriamampita, C., Romao, M., Raposo, G., & Trautmann, A. (2005) Eur J Immunol 35, 1741-1753.
32. Odom, T. W., Love, J. C., Wolfe, D. B., Paul, K. E., & Whitesides, G. M. (2002) Langmuir 18, 5314-5320.
33. Schmid, H. & Michel, B. (2000) Macromolecules 33, 3042-3049.
34. Kung, L. A., Kam, L., Hovis, J. S., & Boxer, S. G. (2000) Langmuir 16, 6773-6776.
35. Sparwasser, T., Gong, S., Li, J. Y., Eberl, G., Sparwasser, T., Gong, S., Li, J. Y. H., & Eberl, G. (2004) Genesis 38, 39-50.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A device for modulating T cells comprising a substrate and spatially organized predefined regions, each predefined region comprising signal molecules for modulating T cells, wherein the spatial organization of the predefined regions is optimized for modulation of T cell expansion, selection and/or activation.

2. The device of claim 1, wherein at least one first predefined region has signal molecules that can activate a T cell receptor.

3. The device of claim 2, wherein at least one second predefined region has signal molecules that can activate CD28 or CD40L.

4. The device of claim 3, wherein a series of the second predefined regions surround, or are interspersed amongst, a series of the first predefined regions.

5. The device of claim 1, wherein the signal molecules are selected from the group consisting of major histocompatibility I proteins, major histocompatibility I peptides, major histocompatibility II proteins, major histocompatibility II peptides, CCL21 (SLC/6Ckine), CD80, CD86, cytokines, B7, Shc-Grb2-SOS, antigens, antigenic peptides, self polypeptides, self peptides, antibodies that bind to T cell surface antigens, or a combination thereof.

6. The device of claim 4, wherein the antibodies that bind to T cell surface antigens bind specifically to a T cell receptor alpha product, T cell receptor beta gene product, CD3, CD28, CD80, CD86, TrimCTLA-4, or ICOS.

7. The device of claim 4, where the cytokines are selected from interleukin-2, interleukin-12 and a combination thereof.

8. The device of claim 1, where the signal molecule is a self peptide or self antigen from the same individual as the T cells.

9. The device of claim 1, where the signal molecule is an antigen or antigenic peptide that can stimulate the T cells.

10. The device of claim 1, where each predefined region separately has one type of signal molecule so that a pattern of segregated predefined regions is present on the device.

11. The device of claim 9, wherein predefined regions with a first signal molecule are surrounded by or interspersed amongst predefined regions with a second and/or third type of signal molecule.

12. The device of claim 1, where one or more predefined regions has a mixture of different types of signal molecules.

13. The device of claim 1, wherein the substrate is silicon, glass, plastic, metal, fibrous, membrane or a combination thereof.

14. The device of claim 1, wherein the substrate comprises a chip, slide, coverslip, plate, petri dish, microtiter well, flask, cylinder, particle, bead, channel, pore, crevice, protuberance or a combination thereof.

15. The device of claim 1, wherein the predefined regions have an area of about 1 μm² to about 200 μm².

16. The device of claim 1, wherein the predefined regions have a signal molecule density of about 100 to about 300 molecules/μm².

17. The device of claim 1, wherein the predefined regions are spaced about 0.1 to about 10 microns apart.

18. The device of claim 1, wherein the predefined regions are spaced about 1 to 2 microns apart.

19. A method for modulating T cells comprising: incubating the T cells on a substrate comprising spatially organized predefined regions, each predefined region comprising signal molecules for modulating T cells, to thereby generate modulated T cells; wherein the spatial organization of the predefined regions is optimized for modulation of T cell expansion, selection and/or activation.

20. The method of claim 19, wherein T cells to be modulated are naïve T cells.

21. The method of claim 19, wherein T cells to be modulated are obtained from bone marrow, thymus, blood, lymph or lymph nodes.

22. The method of claim 19, wherein the T cells are expanded or activated.

23. The method of claim 19, wherein the T cells are activated to recognize a specific antigen.

24. The method of claim 23, wherein the specific antigen is a cancer antigen, viral antigen, bacterial antigen or fungal antigen.

25. The method of claim 19, wherein the modulated T cells are helper (effector) T cells, cytotoxic T cells, memory T cells, effector T cells, regulatory T cells, natural killer T cells, γδ T cells or autoaggressive T cells

26. The method of claim 19, wherein the modulated T cells are memory T cells or helper (effector) T cells.

27. The method of claim 19, wherein at least one first predefined region has signal molecules that can activate a T cell receptor.

28. The method of claim 27, further comprising at least one second predefined region has signal molecules that can activate CD28 or CD40L.

29. The method of claim 28, wherein a series of the second predefined regions surround, or are interspersed amongst, a series of the first predefined regions.

30. The method of claim 19, wherein the signal molecules are selected from the group consisting of major histocompatibility I proteins, major histocompatibility I peptides, major histocompatibility II proteins, major histocompatibility II peptides, CCL21 (SLC/6Ckine), CD80, CD86, cytokines, B7, Shc-Grb2-SOS, antigens, antigenic peptides, self polypeptides, self peptides, antibodies the bind to T cell surface antigens, or a combination thereof.

31. The method of claim 30, wherein the antibodies that bind to T cell surface antigens bind specifically to a T cell receptor alpha product, T cell receptor beta gene product, CD3, CD28, Lck, ZAP-70, CD80, CD86, Trim, LAT, SLP-76, VAV1, Itk, PI(4,5)P2, LFA-1, CTLA-4, or ICOS.

32. The method of claim 24, where the cytokines are selected from interleukin-2, interleukin-12 and a combination thereof.

33. The method of claim 19, wherein the signal molecule is a self peptide or self antigen from the same individual as the T cells.

34. The method of claim 19, where the signal molecule is an antigen or antigenic peptide that can stimulate the T cells.

35. The method of claim 16, wherein each predefined region separately has one type of signal molecule so that a pattern of segregated predefined regions is present on the device.

36. The method of claim 35, wherein predefined regions with a first signal molecule are surrounded by or interspersed amongst predefined regions with a second and/or third type of signal molecule.

37. The method of claim 19, wherein one or more predefined regions has a mixture of different types of signal molecules.

38. The method of claim 19, wherein the substrate is silicon, glass, plastic, metal, fibrous, membrane or a combination thereof.

39. The method of claim 19, wherein the substrate comprises a chip, slide, coverslip, plate, petri dish, microtiter well, flask, cylinder, particle, bead, channel, pore, crevice, protuberance or a combination thereof.

40. The method of claim 19, wherein the predefined regions have an area of about 1 μm to about 200 μm².

41. The method of claim 19, wherein the predefined regions have a signal molecule density of about 100 to about 300 molecules/μm².

42. The method of claim 19, wherein the predefined regions are spaced about 0.1 to about 10 microns apart.

43. The method of claim 19, wherein the predefined regions are spaced about 1 to 2 microns apart.

44. A method of treating an animal comprising administering to the animal a composition of T cells generated by the method of 19 to thereby treat the animal.

45. The method of claim 44, wherein the T cell sample is from the animal.

46. The method of claim 44, wherein T cell sample comprises naïve T cells.

47. The method of claim 44, wherein T cell sample is obtained from bone marrow, thymus, blood, lymph or lymph nodes.

48. The method of claim 44, wherein the T cells are expanded or activated.

49. The method of claim 44, wherein the T cells are activated to recognize a specific antigen.

50. The method of claim 44, wherein the specific antigen is a cancer antigen, viral antigen, bacterial antigen or fungal antigen.

51. The method of claim 44, wherein the modulated T cells are helper (effector) T cells, cytotoxic T cells, memory T cells, effector T cells, regulatory T cells, natural killer T cells, γδ T cells or autoaggressive T cells

52. The method of claim 44, wherein the modulated T cells are memory T cells or helper (effector) T cells.

53. The method of claim 44, wherein the animal has cancer.

54. The method of claim 53, wherein the animal is infected with a virus, bacteria or fungus.

55. The method of claim 53, wherein the animal is infected with an immunodeficiency virus.

56. The method of claim 55, wherein the virus is human immunodeficiency virus.

57. The method of claim 44, wherein the animal is has an immune deficiency.

58. The method of claim 44, wherein the composition is an enriched population of memory or effector T cells

59. A composition comprising an enriched population of memory or effector T cells generated by method comprising incubating a sample of T cells on a substrate comprising spatially organized predefined regions, each predefined region comprising signal molecules for modulating T cells, to thereby generate the enriched population of T cells; wherein the spatial organization of the predefined regions is optimized for T cell expansion and generation of memory or effector T cells.
